SEQUENCE LISTING (10 sequences)

SEQ ID NO: 1 - DNA, 1104 bp, Homo sapiens
  CDS (1)..(1104)
atggtccttgaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttg

SEQ ID NO: 2 - protein, 368 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 3 - DNA, 294 bp, Homo sapiens
  CDS (1)..(294)
atgaatcaaactgcgattctgatttgctgccttatctttctgactctaagtggcattcaaggagtacctctctctagaaccgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaatgtctaaaagatctcct

SEQ ID NO: 4 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKEMSKRSP

SEQ ID NO: 5 - DNA, 375 bp, Homo sapiens
  CDS (1)..(375)
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactaca

SEQ ID NO: 6 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 7 - DNA, 282 bp, Homo sapiens
  CDS (1)..(282)
atgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttt

SEQ ID NO: 8 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 9 - DNA, 327 bp, Homo sapiens
  CDS (1)..(327)
atgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccc

SEQ ID NO: 10 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

(19) United States
(12) Patent Application Publication
(10) Pub. No.: US 2005/0119174 A1
(43) Pub. Date: Jun. 2, 2005

(54) Novel blood sugar controller and method of screening the same

(75) Inventors: Eiji Sugaru, Osaka-shi (JP); Junji Ichihara, Ibaraki-shi (JP); Mutsuo Taiji, Takatsuki-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Sumitomo Pharmaceuticals Co. Ltd., Osaka (JP)

(21) Appl. No.: 10/510,121

(22) PCT Filed: Apr. 3, 2003
(86) PCT No.: PCT/JP03/04260

(30) Foreign Application Priority Data
Apr. 3, 2002 (JP) 2002-101781

(51) Int. Cl. C12Q 1/68; G01N 33/53; G01N 33/567; A61K 38/22

(52) U.S. Cl. 514/12; 530/399; 536/23.5; 435/6; 435/7.2

(57) ABSTRACT

The present invention provides an impaired glucose tolerance ameliorating drug and a therapeutic drug for lifestyle-related diseases, particular an antidiabetic drug, which contain a CXCR3 agonist as an active ingredient; a hypoglycemia ameliorating drug, a therapeutic drug for insulinoma or an anti-obesity drug containing a CXCR3 antagonist as an active ingredient; a method of screening for a new CXCR3 ligand using CXCR3 and a known CXCR3 ligand; a method of screening a CXCR3 ligand, which uses a co-expression system of CXCR3 and a coupling G protein; and a diagnostic method for type II diabetes, which includes detecting an amount of a CXCR3 ligand expressed in a biological sample.

### TECHNICAL FIELD

The present invention relates to new pharmaceutical use of CXCR3 ligands. More specifically, the present invention relates to use of CXCR3 ligand for insulin secretion regulation, for example, use of CXCR3 agonist for impaired glucose tolerance amelioration and the treatment of lifestyle-related diseases such as diabetes, and use of CXCR3 antagonist for the amelioration of hypoglycemia and the treatment of diseases associated with insulin hypersecretion, such as obesity. The present invention also relates to a novel screening method for a CXCR3 ligand capable of being a therapeutic drug for the above-described diseases. The present invention still also relates to a diagnostic method for type II diabetes comprising examining the expression of a physiological ligand for CXCR3 in a biological sample and a laboratory test reagent for the same.

### BACKGROUND ART

Diabetes is classified into type I and type II according to the pathologic condition thereof. That is, type I is a pathologic condition based on insulin secretion dysfunction in the pancreas, and type II is a pathologic condition mainly involving insulin resistance in insulin-sensitive tissue and impaired insulin secretion in the pancreas. In recent years, due to dietary life westernization, increased social stress and the like, patients with obesity and accompanying lifestyle-related diseases, particularly type II diabetes patients, have been increasing dramatically.

In blood glucose regulation, the pancreas is considered to play a central role. Insulin, a major blood glucose regulation hormone, is secreted from the β cells of the pancreatic islet (of Langerhans). The β cells quickly secrete a necessary amount of insulin in response to a transient elevation of blood glucose postprandially and the like. In peripheral tissues such as muscles and adipose, elevated blood glucose is regulated by incorporating sugar in response to the insulin secreted from the pancreas. Furthermore, in the liver, gluconeogenesis is suppressed in response to insulin, and blood glucose is regulated. It is considered that diabetes develops as a result of breakage in this cycle. In fact, in many cases of type I diabetes, blood glucose control becomes impossible due to autoimmune destruction of the pancreas and insulin secretion insufficiency, and it is known that in type II diabetes, poor secretion in the first phase of insulin secretion causes hyperglycemia.

From the viewpoint described above, insulin secretion promotion in the pancreas is considered to contribute significantly to ameliorating effects on the pathologic condition of diabetes. As drugs having such an action mechanism, sulfonylurea agents and the like are currently available in the market; however, due to the lack of insulin secretion regulating action according to blood glucose level, the insulin secretion promotion effect persists to cause hypoglycemia, even after blood glucose decreases, which can lead to the risk of coma, and even of death. At present, no safe drugs characterized by the obtainment of an insulin secretion promotion effect according to blood glucose level are available in the market; the development of such a drug is demanded.

Accordingly, the object of the present invention is to provide a compound possessing sugar metabolism regulating action and a method of screening the same, and to provide a method of ameliorating impaired glucose tolerance using them and a therapeutic drug for diabetes and other lifestyle-related diseases.

### DISCLOSURE OF THE INVENTION

The present inventors, in an attempt to identify a factor involved in the regulation of insulin secretion in the pancreas, compared genes expressed in the normal human pancreas and genes expressed in other normal human tissues, and, as a result, identified, as a gene expressed locally in the normal human pancreas, the gene for CXC chemokine receptor type 3 (CXCR3; or also called GPR9), which is a kind of chemokine receptor. As a result of an investigation of more detailed localization of this receptor in the pancreas, this protein proved to be specifically expressed in the islet of Langerhans, and was strongly suggested as being involved in insulin secretion regulation.

Chemokine is a generic name for a series of cytokines discovered as governing the migratory action and activation of a particular subset of leukocytes. Chemokines have cysteine residues conserved in their molecules, and are classified into four subfamilies according to the position thereof on the molecular structure: CXC, CC, C and CXXXC. As functions of chemokines, roles as mediators for the infiltration of specific leukocytes in immunization and inflammation but nothing has been known to date about their actions on the control of sugar metabolism.

The present inventors next investigated whether or not CXCR3-mediated signal transmission is involved in insulin secretion in the pancreas, and whether or not CXCR3 ligands exhibit pancreas-mediated antidiabetic action. That is, the present inventors evaluated the effects of IP-10, Mig and I-TAC, which are chemokines having CXCR3 as the receptor, on impaired glucose tolerance in diabetic model mice, and found that these chemokines transiently promote insulin secretion in response to increased blood glucose levels arising from glucose loading, and exhibit a remarkable impaired glucose tolerance ameliorating action. The changes in blood glucose level due to IP-10 administration were similar to those with glucagon-like peptide-1 (GLP-1), which exhibits incretin action. Because GLP-1 has been reported to be involved in eating control, IP-10 is also expected to have an effect to regulate food consumption, while controlling blood glucose level, and was suggested as being also effective in the treatment of obesity due to bulimia and various accompanying lifestyle-related diseases.

These results show that not only chemokines which are physiological ligands for CXCR3, such as IP-10, Mig and I-TAC, as well as recently reported BCA-1 (CXCL13), but also all compounds possessing agonist activity against CXCR3 possess impaired glucose tolerance ameliorating action and therapeutic activity for lifestyle-related diseases including diabetes. On the other hand, because compounds possessing antagonist activity against CXCR3 are capable of regulating insulin secretion by suppressing the abnormally increased CXCR3-mediated signal transmission, they are considered to exhibit ameliorating action on hypoglycemia and other various pathologic conditions expected to be ameliorated by insulin secretion suppression, anti-obesity action and the like. Accordingly, the provision of a means of searching for a new ligand for CXCR3 is very important in the development of therapeutic drugs for the above-described diseases.

Thus, the present inventors succeeded in constructing a screening system for a novel CXCR3 ligand using CXCR3 or a cell that expresses the same and a known CXCR3 ligand, which resulted in the completion of the present invention.

That is, the present invention provides a blood glucose and/or insulin secretion regulator comprising a CXCR3 ligand as an active ingredient, specifically an impaired glucose tolerance ameliorating drug and a therapeutic drug for lifestyle-related diseases, particularly for diabetes, which comprises a CXCR3 agonist as an active ingredient, and a hypoglycemia ameliorating drug, a therapeutic drug for diseases that can be ameliorated by suppression of insulin secretion, and an anti-obesity drug comprising a CXCR3 antagonist as an active ingredient.

Another embodiment of the present invention provides a screening method for a CXCR3 ligand, which comprises bringing a test sample into contact with CXCR3 or a fragment thereof to which a ligand can bind, and selecting a compound that binds to said receptor or a fragment thereof.

Still another embodiment of the present invention provides a screening method for a CXCR3 ligand, which comprises bringing a known ligand into contact with CXCR3 or a fragment thereof to which a ligand can bind, in the presence and absence of a test substance, and comparing the binding activity of said receptor or a fragment thereof and the known ligand under both conditions.

CXCR3 is a trimeric GTP-binding protein coupled receptor (GPCR), and some findings have been obtained with respect to the coupling G protein α subunit (Gα). Accordingly, the present invention provides a screening method for a CXCR3 ligand, which comprises comparing, in a reaction system containing a CXCR3-containing lipid bilayer and coupling Gα, the GDP-GTP exchange reaction of said Gα or the cell stimulating activity of coupling G protein, in the presence and absence of a test substance.

Still yet another embodiment of the present invention provides a blood glucose and/or insulin secretion regulator comprising a CXCR3 ligand selected by any one of the above-described screening methods as an active ingredient, specifically an impaired glucose tolerance ameliorating drug and therapeutic drug for lifestyle-related diseases, particularly for diabetes, comprising a CXCR3 agonist as an active ingredient, and a hypoglycemia ameliorating drug, therapeutic drug for diseases that can be ameliorated by insulin secretion suppression, and anti-obesity drug comprising a CXCR3 antagonist as an active ingredient.

Based on the finding of the present invention, because substances that enhance the expression or activity of CXCR3, in addition to CXCR3 agonists, are capable of promoting signal transmission mediated by said receptor, they are effective in the amelioration of impaired glucose tolerance and the treatment of lifestyle-related diseases, particularly of diabetes. Accordingly, the present invention also provides an impaired glucose tolerance ameliorating drug and a therapeutic drug for lifestyle-related diseases, particularly for diabetes, which comprises a substance that enhances the expression or activity of CXCR3, other than CXCR3 agonists, as an active ingredient.

On the other hand, because substances that inhibit the expression or activity of CXCR3, in addition to CXCR3 antagonists, are capable of blocking signal transmission mediated by said receptor, they exhibit ameliorating action on hypoglycemia and other various pathologic conditions expected to be ameliorated by insulin secretion suppression, anti-obesity action and the like. Accordingly, the present invention also provides a hypoglycemia ameliorating drug, therapeutic drug for diseases that can be ameliorated by insulin secretion suppression, and anti-obesity drug comprising a substance that inhibits the expression or activity of CXCR3, other than CXCR3 antagonists, as an active ingredient.

Also, in type II diabetes patients, the blood concentration of physiological ligands for CXCR3 has increased significantly compared to healthy people. Accordingly, the present invention also provides a diagnostic method for type II diabetes comprising measuring a CXCR3 ligand or the transcript of the gene for said ligand in a biological sample using an antibody possessing specific affinity for the physiological ligand for CXCR3 or a nucleic acid that encodes said ligand or a nucleic acid hybridizable with said nucleic acid under stringent conditions, and a diagnostic reagent for type II diabetes containing said antibody or said nucleic acid.

Further characteristics of the present invention and advantages of the present invention are described in more detail in the “Modes of Embodiment of the Invention” below.

### BRIEF DESCRIPTION OF THE DRAWINGS

FIG. 1 is an electrophoresis image showing the localized expression of CXCR3 in the mouse pancreatic islet. Soluble fractions of the Langerhans island and whole pancreas (12 μg of each) derived from ICR mice were each separated by SDS-PAGE, and subjected to Western blotting using an anti-CXCR3 antibody as a probe. The band position corresponds to about 40.3 KDa.

FIG. 2 shows the effects of IP-10 and GLP-1 administrations on time course changes of blood glucose level after glucose loading in mice. The abscissa indicates time after glucose loading, and the ordinate indicates blood glucose level (mg/dl), wherein ♦ indicates the solvent control group, ▪ indicates the IP-10 administration group and ▴ indicates the GLP-1 administration group.

FIG. 3 shows the area under curve of the blood glucose level until 135 minutes after glucose loading (AUC; mg^{x}135 minutes/dl) for each administration group in FIG. 2.

FIG. 4 shows the effects of Mig and I-TAC administrations (intravenous administrations) on changes in blood glucose level at 90 minutes after glucose loading in mice. The ordinate indicates blood glucose level (mg/dl).

FIG. 5 shows the responsiveness of the CHO/Gqi5 cell line allowed to transiently express CXCR3 (upper graph) to IP-10 (FIG. 5B) and I-TAC (FIG. 5A). The lower graph shows the results for the CHO/Gqi5 cell line incorporating an empty vector (pcDNA3.1). In each graph, the abscissa indicates time (seconds) and the ordinate indicates changes in fluorescence intensity, wherein -▪- shows the results for a ligand concentration of 300 ng/ml and -*- shows the results for the buffer alone.

FIG. 6 shows the responsiveness of the CHO/Gqi5 cell line that stably expresses CXCR3 (#122-17) to IP-10 (FIG. 6A) and I-TAC (FIG. 6B). In each graph, the abscissa indicates time (seconds) and the ordinate indicates changes in fluorescence intensity, wherein ♦ shows the results for ligand concentration of 1,000 ng/ml, ▪ shows the results for 300 ng/ml and ▴ shows the results for 100 ng/ml, and * shows the results for the buffer alone.

### BEST MODE FOR EMBODYING THE INVENTION

“CXCR3” is a shared receptor for IP-10, Mig, I-TAC and BCA-1, which are CXC chemokines, and is a GPCR known to be specifically expressed on the monocyte or Th1 cell surface. The base sequence of the CXCR3 gene and the amino acid sequence of CXCR3 are known; for example, the base sequence of human CXCR3 cDNA (SEQ ID NO:1) has been registered with GenBank under accession number NM_{—}001504. In the present invention, “CXCR3” is not subject to limitation, as long as it is capable of interacting with a ligand such as IP-10, Mig, I-TAC or BCA-1, and is used with the meaning encompassing, in addition to CXCR3s derived from human and other mammals, all of natural or artificial mutants thereof, recombinant CXCR3s produced from recombinant cells containing a DNA that encodes the same, and functional fragments thereof.

More specifically, the CXCR3 of the present invention refers to a protein containing the amino acid sequence shown by SEQ ID NO: 2 or an amino acid sequence substantially the same as it, and possessing an activity of the same nature as the protein consisting of the amino acid sequence shown by SEQ ID NO:2. “Substantially the same” as used herein refers to possessing about 70% or higher, preferably about 80% or higher, more preferably about 90% or higher, amino acid identity when a homology search program conventionally used in the relevant technical field (for example, BLAST, FASTA and the like) is used. Also, “activity of the same nature” means that the activity is qualitatively equivalent; although the activity is preferably also quantitatively equivalent, it may differ within an acceptable range (for example, about 0.5 to about 2 times). Here, as the activity of CXCR3, ligand binding activity and cell stimulating activity (for example, intracellular calcium concentration increasing activity, intracellular cAMP concentration decreasing activity and the like) can be mentioned.

“CXCR3 ligand”, unless otherwise specified, is understood to encompass not only physiological ligands (IP-10, Mig, I-TAC, BCA-1) but also agonists (that is, substances that bind to the physiological ligand binding sites of receptors to exhibit ligand-like activity), antagonists (substances that bind to the physiological ligand binding sites of receptors but do not exhibit ligand-like activity) and inverse agonists (substances that bind to any sites of receptors to alter their conformations and to inactivate the receptors). For example, eotaxin, MCP-3, MCP-4, RANTES, MIP-3a, and 6Ckine, which are CC chemokines, are known to weakly bind to CXCR3 [J. Biol. Chem., 273(29): 18288-18291 (1998); Eur. J. Immunol., 29: 3804-20 3812 (1999)].

“CXCR3 agonist” is used as a generic name for substances that bind to CXCR3 to promote the activity of said receptor, and encompasses, in addition to IP-10, Mig, I-TAC, and BCA-1, which are physiological ligands for CXCR3, and modifications thereof, all known and novel compounds that possess agonist activity against CXCR3. Preferably, said agonist is selected from a group consisting of IP-10, Mig, I-TAC, BCA-1, modifications thereof and prodrugs thereof (prodrugs hereunder described), particularly preferably selected from a group consisting of IP-10, Mig, I-TAC, modifications thereof and prodrugs thereof (prodrugs hereunder described).

All of the amino acid sequences of IP-10, Mig, I-TAC and BCA-1, which are physiological ligands for CXCR3, and the base sequences of their genes are known; for example, the cDNA sequences of these ligands derived from the human have been registered with GenBank under accession numbers NM_{—}001565 (SEQ ID NO:3), NM_{—}002416 (SEQ ID NO:5), NM_{—}005409 (SEQ ID NO:7), and NM_{—}006419 (SEQ ID NO:9), respectively. In the present invention, “IP-10”, “Mig”, “I-TAC”, and “BCA-1” encompass, in addition to these chemokine proteins derived from the human and other mammals, recombinant proteins produced from recombinant cells containing DNAs that encode them.

“Modifications” of IP-10, Mig, I-TAC and BCA-1 encompass all peptide substances which comprise the amino acid sequences of mature proteins of these chemokines derived from the human and other mammals (for example, the amino acid sequence of amino acid number 1 and beyond in the amino acid sequences shown by SEQ ID NO:4, 6, 8 and 10) with one or a plurality of amino acids substituted, deleted, inserted, added or modified, and which retain agonist activity against CXCR3. “A plurality of amino acids” are preferably 1˜30 amino acids, more preferably 1˜10 amino acids, still more preferably 1˜5 amino acids, and these may be continuous or uncontinuous. For example, natural or artificial mutants of IP-10, Mig, I-TAC and BCA-1 derived from optionally chosen mammals, functional fragments of IP-10, Mig, I-TAC and BCA-1 derived from optionally chosen mammals, and the like are also included in the “modifications” in the present invention.

Physiological ligands for CXCR3, like GLP-1, which exhibits insulin secretion promoting action, are susceptible to degradation by dipeptidyl peptidase IV (DPPIV), and the degradation products thereof are known to retain CXCR3-binding activity but lose chemokine activity. That is, because said degradation products act as CXCR3 antagonists to diminish the glucose tolerance ameliorating action of intact ligands, it is more desirable that DPPIV resistance be conferred to these ligands. Accordingly, as preferable modifications, those resulting from cyclization or lecithination of these ligands, those resulting from substitution of the second proline from the N-terminus with another amino acid, and the like can be mentioned. Such peptide modifications can easily be conducted using a technique known per se.

CXCR3 agonists, in recipient animals, transiently promote insulin secretion in response to increased blood glucose levels and reduce blood glucose levels; because insulin secreting action decreases after the reduction in blood glucose level, CXCR3 agonists do not cause hypoglycemia and are effective as safe impaired glucose tolerance ameliorating drugs and therapeutic drugs for diabetes.

Also, because CXCR3 agonists have effects similar to those of GLP-1, which is known to be involved in eating control, they are also effective as therapeutic drugs not only for diabetes but also for obesity caused by eating abnormalities such as bulimia and various accompanying lifestyle-related diseases.

“CXCR3 antagonists” are used as a generic term for substances which bind to CXCR3 but do not promote the activity of said receptor, and are divided into neutral antagonists, which block the activity of CXCR3 by inhibiting the binding of physiological ligands to CXCR3, and inverse agonists, which shift the equilibrium of the active form and inactive form of CXCR3 toward the more inactive side. CXCR3 antagonists encompass all known and novel compounds that possess any one of the above-described properties; for example, degradation products of IP-10, Mig, I-TAC, and BCA-1, which are physiological ligands for CXCR3, with the above-described DPPIV, certain kinds of CC chemokines known to possess affinity for CXCR3 (for example, eotaxin has been suggested as being an antagonist for CXCR3 [J. Biol. Chem., 273(29): 18288-18291 (1998)]) and the like can be mentioned.

Because CXCR3 antagonists transiently suppress insulin secretion in response to blood glucose level reductions and elevate blood glucose levels in recipient animals, they are effective as hypoglycemia ameliorating drugs, therapeutic drugs for other diseases that can be ameliorated by insulin secretion suppression (for example, insulinoma and the like), and anti-obesity drugs.

CXCR3 ligands can also be used in the form of a prodrug that is metabolized in the bodies of recipient animals and exhibits ligand activity against CXCR3 (agonist activity or antagonist activity). When the CXCR3 ligand is a peptide substance such as IP-10, Mig, I-TAC or BCA-1, “prodrugs” thereof also encompass an expression vector containing a DNA that encodes the peptide, and a host cell transfected with said expression vector.

In the expression vector, a DNA that encodes a physiological ligand possessing agonist activity against CXCR3, such as IP-10, Mig, I-TAC or BCA-1, or a modification thereof, or a peptide possessing antagonist activity against CXCR3, such as DDPIV degradation products thereof, must be operably linked to a promoter capable of exhibiting promoter activity in the cells of the recipient mammal, or arranged at a position such that said DNA is capable of turning into an operably linked form under particular conditions in the cells of the recipient animal.

As the DNA that encodes a physiological ligand possessing agonist activity against CXCR3, such as IP-10, Mig, I-TAC or BCA-1, a DNA that encodes the amino acid sequence shown by SEQ ID NO: 4, 6, 8 or 10, preferably a DNA containing the base sequence shown by SEQ ID NO:3, 5, 7 or 9 can be mentioned. Also, as the DNA that encodes a modification of said physiological ligand, a DNA that encodes a polypeptide which contains the base sequence that encodes the above-described amino acid sequence with one or a plurality of amino acids (for example, 1˜30 amino acids, preferably 1˜10 amino acids, more preferably 1˜5 amino acids) substituted, deleted, inserted or added, and which retains agonist activity against CXCR3 can be mentioned.

Although the promoter used is not subject to limitation, as long as it is capable of functioning in the cells, preferably liver, pancreas and small intestine, of the recipient mammal; for example, viral promoters such as the SV40-derived early promoter, cytomegalovirus LTR, Rous sarcoma virus LTR, MoMuLV-derived LTR and adenovirus-derived early promoter, and mammalian constitutive protein gene promoters such as the β-actin gene promoter, PGK gene promoter and transferrin gene promoter can be mentioned. “Arranged at a position such that . . . is capable of turning into an operably linked form under particular conditions” refers to, for example, as described in more detail below, that the promoter and the DNA that encodes a CXCR3 ligand are separated by a spacer sequence sufficiently long to prevent the expression of said CXCR3 ligand from said promoter, split by two recombinase recognition sequences arranged in the same direction, said spacer sequence is cleared out in the presence of a recombinase that specifically recognizes said recognition sequence, and the DNA that encodes said CXCR3 ligand is operably linked to the promoter.

The expression vector of the present invention preferably contains a transcription termination signal, that is, a terminator region, downstream of the DNA that encodes a CXCR3 ligand. Furthermore, the expression vector of the present invention can further contain a selection marker gene for transformed cell selection (a gene that confers resistance to drugs such as tetracycline, ampicillin, kanamycin, hygromycin and phosphinothricin, a gene that complements auxotrophic mutations, and the like). When the expression vector has a spacer sequence sandwiched between recombinase recognition sequences as described above, said selection marker gene can also be arranged in the spacer sequence. Also, when the DNA that encodes a CXCR3 ligand has a base sequence that encodes a signal sequence (signal codon) (for example, the base sequence that encodes the amino acid sequence of amino acid number-1 and beyond in the amino acid sequence shown by SEQ ID NO:4, 6, 8 or 10), the sequence may be replaced with another signal codon.

Although the vector used for the expression vector of the present invention is not subject to limitation, as vectors suitable for administration to mammals such as the human, viral vectors such as retrovirus, adenovirus, adeno-associated virus, herpes virus, vaccinia virus, pox virus, polio virus, Sindbis virus and Sendai virus can be mentioned. Adenovirus has advantages such as extremely high gene transfection efficiency and transfectability to non-dividing cells as well. However, because the incorporation of the transfected gene in the host chromosome is extremely rare, the gene expression is transient and usually lasts only for about 4 weeks. Considering the persistency of therapeutic effect, use of adeno-associated virus, which offers relatively high gene transfection efficiency, which is transfectable to non-dividing cells as well, and which can be incorporated in chromosome via an inverted terminal repeat sequence (ITR), is also preferable.

In a mode of embodiment of the present invention, the expression vector enables the time-specific and/or tissue-specific expression of a CXCR3 ligand to prevent the adverse effects of the overexpression of the CXCR3 ligand at an unwanted time and/or unwanted site. As a first mode of embodiment of such a vector, a vector containing a DNA that encodes a CXCR3 ligand operably linked to a promoter derived from a gene that specifically expresses in the CXCR3-ligand-producing cells of the recipient animal can be mentioned. For example, the native promoter of the gene of a physiological ligand possessing agonist activity against CXCR3, such as IP-10, Mig, I-TAC or BCA-1, and the like can be mentioned.

As a second mode of embodiment of the time-specific and tissue-specific expression vector of the present invention, a vector containing a DNA that encodes a CXCR3 ligand operably linked to an inducible promoter whose expression is controlled in trans by an exogenous substance can be mentioned. When the metallothionein-1 gene promoter, for example, is used as the inducible promoter, the expression of a CXCR3 ligand can be induced tissue-specifically at an optionally chosen time by topically administering an inducer such as a heavy metal such as gold, zinc or cadmium, a steroid such as dexamethasone, an alkylating agent, a chelating agent or a cytokine to the desired tissue (for example, liver, pancreas, small intestine and the like) at the desired time.

Another preferred embodiment of the time-specific and tissue-specific expression vector of the present invention is a vector having a structure wherein the promoter and the DNA that encodes a CXCR3 ligand are separated by a spacer sequence sufficiently long to prevent the expression of said ligand from said promoter, split by two recombinase recognition sequences arranged in the same direction. Solely transfecting said vector into the target cell does not ensure that the promoter directs the transcription of the CXCR3 ligand. However, provided that a recombinase that specifically recognizes said recognition sequences is topically administered to the target tissue at the desired time, or an expression vector containing a DNA that encodes said recombinase is topically administered to express said recombinase in the target cell, homologous recombination via said recombinase occurs between said recognition sequences; as a result, said spacer sequence is cleared out, the DNA that encodes the CXCR3 ligand is operably linked to the promoter, and the CXCR3 ligand is expressed tissue-specifically at the desired time.

It is desirable that the recombinase recognition sequences used for the above-described vector be heterologous recombinase recognition sequences not recognized by endogenous recombinase, to prevent recombination by the recombinase that is endogenous to the recipient. Therefore, it is desirable that the recombinase that trans-acts on said vector be also a heterologous recombinase. As such combinations of a heterologous recombinase and said recombinase recognition sequences, Escherichia coli bacteriophage P1-derived Cre recombinase and the loxP sequence, or yeast-derived Flp recombinase and the frt sequence can preferably be mentioned but are not to be construed as limiting.

Discovered in a bacteriophage, Cre recombinase is known to work in the specific DNA recombination reaction, not only in prokaryotic cells but also in animal cells and animal viruses, which are eukaryotic cells. When two lox P sequences are present on the same DNA molecule in the same direction, Cre recombinase cleaves out the DNA sequence sandwiched by the sequences to allow them to form a cyclic molecule (cleavage reaction). On the other hand, in cases where two lox P sequences are present on different DNA molecules one of which is cyclic DNA, the cyclic DNA is inserted to the other DNA molecule via the lox P sequence (insertion reaction) [J. Mol. Biol., 150: 467-486 (1981); J. Biol. Chem., 259: 1509-1514 (1984); Proc. Natl. Acad. Sci. USA, 81: 1026-1029 (1984)]. Example cleavage reactions are reported in animal cells in culture [Nucleic Acids Res., 17: 147-161 (1989); Gene, 181: 207-212 (1996)], animal viruses [Proc. Natl. Acad. Sci. USA, 85: 5166-5170 (1988); J. Virol., 69: 4600-4606 (1995); Nucleic Acids Res., 23: 3816-3821 (1995)], transgenic mice [Proc. Natl. Acad. Sci. USA, 89: 6232-6236 (1992); Proc. Natl. Acad. Sci. USA, 89: 6861-6865 (1992); Cell, 73: 1155-1164 (1993); Science, 265:103-106 (1994)], etc.

As the promoter for the time-specific and tissue-specific expression vector of the present invention, which utilizes the recombinase/recombinase recognition sequence interaction, a virus-derived promoter or a mammalian constitutive protein gene promoter is preferably used to ensure expression at the desired time and tissue.

Administration of the expression vector containing a DNA that encodes a CXCR3 ligand is conducted by either the ex vivo method, wherein cells that produce the physiological ligand for CXCR3 of the treatment subject animal are taken out from the body, cultured, and then the vector is transfected and the cells are returned into the body, or the in vivo method, wherein the vector is administered directly into the body of the recipient to achieve its transfection. In the case of the ex vivo method, vector transfection to the target cell can be conducted by the microinjection method, calcium phosphate co-precipitation method, PEG method, electroporation method and the like. In the case of the in vivo method, the viral vector is administered in the form of an injection and the like intravenously, intra-arterially, subcutaneously, intracutaneously, intramuscularly, intraperitoneally and the like. Alternatively, when the vector is administered by intravenous injection and the like, production of a neutralizing antibody against the viral vector becomes problematic; however, provided that the vector is topically injected to the site where the target cell is present (in situ method), the adverse effects of the presence of the antibody can be mitigated.

Also, when a non-viral vector is used as the expression vector containing a DNA that encodes a CXCR3 ligand, transfection of said expression vector can be conducted using a high molecular carrier such as the poly-L-lysine-nucleic acid complex or in a liposome-enveloped state. Alternatively, the vector can also be transfected directly to the target cell using the particle gun method.

In the use of a vector utilizing the recombinase/recombinase recognition sequence interaction, when recombinase itself is topically administered as the transacting substance, the recombinase may, for example, be topically injected on dissolving or suspending in an appropriate sterile vehicle. On the other hand, when a recombinase expression vector is topically administered as the trans-acting substance, said recombinase expression vector is not subject to limitation, as long as it has an expression cassette wherein the recombinase-encoding polynucleotide is operably linked to a promoter capable of exhibiting promoter activity in cells of the recipient animal that produce a physiological ligand for CXCR3. When the promoter used is a constitutive promoter, it is desirable that the vector administered to the target cell be one rarely incorporated in the host cell chromosome, like, for example, adenovirus, to prevent the expression of recombinase at unwanted times. However, when an adenovirus vector is used, the transient expression of recombinase lasts only for at most about 4 weeks; therefore, if the treatment involves a long time, a second and third administration will be necessary. As another approach to expressing recombinase at the desired time, use of an inducible promoter like the metallothionein gene promoter can be mentioned. In this case, use of a viral vector of high integration efficiency, such as retrovirus, is possible.

When the preparation of the present invention has a host cell containing an expression vector containing a DNA that encodes a CXCR3 ligand as an active ingredient, as examples of the host cell used, in the ex vivo transfection method of the expression vector as described above, an autologous cell taken out as the target cell from the recipient, or cells taken out from allogenic (for example, stillborn fetuses, brain-dead patients and the like, in the case of the human) or heterologous (other mammalians such as the swine and the monkey, in the case of the human) individuals, or cells obtained by culturing and differentiating stem cells thereof and ES cells, and the like can be mentioned.

Also, in another embodiment, it is also possible to transform a bacterium or the like that is resident in the nasal cavity, pharynx, oral cavity, intestinal tract and the like of the recipient animal as the host cell, with an expression vector containing a DNA that encodes a CXCR3 ligand, by a conventional method, and to deliver the obtained transformant to a site in the recipient where the host cell is normally present.

The CXCR3 ligand, along with a pharmaceutically acceptable carrier, is prepared as a dosage form suitable for oral or parenteral administration. As examples of the pharmaceutically acceptable carrier, excipients such as sucrose, starch, mannitol, sorbitol, lactose, glucose, cellulose, talc, calcium phosphate and calcium carbonate; binders such as cellulose, methylcellulose, hydroxypropylcellulose, polypropylpyrrolidone, gelatin, acacia, polyethylene glycol, sucrose and starch; disintegrants such as starch, carboxymethylcellulose, hydroxypropyl starch, sodium-glycol-starch, sodium hydrogen carbonate, calcium phosphate and calcium citrate; lubricants such as magnesium stearate, Aerosil, talc and sodium lauryl sulfate; flavoring agents such as citric acid, menthol, glycyrrhizin ammonium salt, glycine and orange flour; preservatives such as sodium benzoate, sodium hydrogen sulfite, methyl paraben and propyl paraben; stabilizers such as citric acid, sodium citrate and acetic acid; suspending agents such as methylcellulose, polyvinylpyrrolidone and aluminum stearate; dispersing agents such as surfactants; diluents such as water, physiological saline and orange juice; base waxes such as cacao butter, polyethylene glycol and refined kerosene; and the like can be mentioned, which, however, are not to be construed as limiting.

Preparations suitable for oral administration are a liquid comprising an effective amount of CXCR3 ligand dissolved in a diluent like water, physiological saline or orange juice, a capsule, sachet or tablet containing an effective amount of CXCR3 ligand as a solid or granules, a suspension comprising an effective amount of CXCR3 ligand suspended in an appropriate dispersion medium, an emulsion comprising a solution of an effective amount of CXCR3 ligand dispersed and emulsified in an appropriate dispersion medium, and the like. Here, “an effective amount” refers to an amount sufficient to ameliorate each disease when a CXCR3 agonist is used for the treatment of an impaired glucose tolerance patient, a diabetes patient or another lifestyle-related disease patient, or an amount sufficient to ameliorate each disease when a CXCR3 antagonist is used for the treatment of a hypoglycemia patient, a patient suffering from another disease that can be ameliorated by insulin secretion suppression or an obesity patient.

As preparations suitable for parenteral administration (for example, intravenous injection, intra-arterial injection, subcutaneous injection, intramuscular injection, topical injection, intraperitoneal administration and the like), aqueous and non-aqueous isotonic sterile injectable liquids are available, which may contain an antioxidant, a buffer solution, a bacteriostatic agent, an isotonizing agent and the like. Also, aqueous and non-aqueous sterile suspensions can be mentioned, which may contain a suspending agent, a solubilizer, a thickener, a stabilizer, an antiseptic and the like.

The CXCR3 ligand preparation can be sealed in a container in a unit dose or a multiple dose like an ampoule or vial. Also, the CXCR3 ligand and pharmaceutically acceptable carrier can also be lyophilized and in a state that only requires dissolving or suspending in an appropriate sterile vehicle just before use.

Although the dose and administration frequency of the CXCR3 ligand preparation vary depending on symptoms, age, body weight, dosage form and the like, the preparation can normally be administered in the range of about 0.0001 to about 500 mg, preferably in the range of about 0.001 to about 100 mg, per day for each adult, at a time or in several divided portions.

Although the dose of the preparation of the present invention with an expression vector that encodes a CXCR3 ligand or a host cell harboring said expression vector as an active ingredient varies depending on the kind of active ingredient, promoter activity, route of administration, seriousness of disease, recipient animal species, drug acceptability, body weight and age of the recipient and the like, the dose is preferably such that the CXCR3 ligand molecule in an amount equivalent to the appropriate dose for the administration of a therapeutic drug with the CXCR3 ligand molecule itself as an active ingredient is expressed in the body of the animal receiving the vector or the host cell, and is, for example, about 2 to about 20 μg/kg, preferably about 5 to about 10 μg/kg, based on the amount of vector per day for each adult.

The present invention also provides a CXCR3 ligand screening system and a screening method for the ligand using it.

A first embodiment of the screening method of the present invention comprises a step bringing a test sample into contact with CXCR3 or a fragment thereof to which a ligand can bind, and a step selecting a compound that binds to said receptor or the fragment thereof. The test sample may be any known compound or a novel compound; for example, a compound library prepared using combinatorial chemistry technology, a random peptide library prepared by solid phase synthesis or the phage display method, or naturally occurring ingredients derived from microorganisms, animals, plants, marine organisms and the like, and the like can be mentioned. Binding activity to the test substance can, for example, be derived by immobilizing a cell membrane fraction that expresses CXCR3 or a fragment thereof on a chip, loading a test sample solution on said chip, measuring the binding to and dissociation from the membrane of the test substance by the surface plasmon resonance method, and calculating the affinity of the test substance and CXCR3 from the binding and dissociation rates or the amount bound.

A second embodiment of the screening method of the present invention comprises bringing a known ligand into contact with CXCR3 or a fragment thereof to which a ligand can bind, in the presence and absence of a test substance, and comparing the binding activity of said receptor or the fragment thereof and the known ligand under both conditions. As the known ligand, IP-10, Mig, I-TAC, BCA-1 and the like, which are physiological ligands for CXCR3, can be used.

In all the above-described embodiments, CXCR3 or a fragment thereof can be provided in the form of a cell that expresses them, the cell membrane fraction of said cell, or a state bound to an affinity column. As CXCR3-expressing cells, cells transfected with an expression vector containing a DNA that encodes CXCR3 or a fragment thereof, monocytes or Th1 cells that endogenously express CXCR3, and the like can be mentioned. Also, as the affinity column, an anti-CXCR3 antibody column, a column using a known ligand, and, when CXCR3 is provided as a recombinant protein, a metal chelate column, glutathione column, biotin-coated column and the like possessing specific affinity for the His tag, the GST tag, (strepto)avidin and the like, respectively, can be used.

Detection of the binding activity of CXCR3 or a fragment thereof and a known ligand can, for example, be conducted by labeling the known ligand, and measuring the amount of label bound to CXCR3 or a fragment thereof.

CXCR3 is a GPCR and transmits signals into cells coupled with a certain kind of trimeric G protein. Accordingly, the present invention also provides a screening method of a CXCR3 ligand using a CXCR3-containing lipid bilayer and a G protein (particularly the Gα-subunit) coupled with CXCR3. The screening method of the present invention is conducted with the GTP-GDP exchange reaction in Gα or the cell stimulating activity of the coupling G protein as the index. When the cell stimulating activity of the G protein is used as the index, specific procedures such as effector selection and assay method are determined according to the type of the coupling Gα; usually, CXCR3 ligands can be screened by, for example, measuring the amount of cAMP when a Gα containing a region that interacts with adenylate cyclase (specifically, containing the region interacting with the effector of a Gα belonging to the Gi or Gs family) is used, and by, for example, measuring the amount of intracellular calcium ions when a Gα containing a region that interacts with phospholipase Cβ (specifically, containing the region inter-acting with the effector of a Gα belonging to the Gq family) is used.

Also, when an animal cell that endogenously expresses a trimeric G protein (Gαβγ) containing said Gα (for example, HEK29 cells, L1.2 cells and the like) is used as the Gα source, CXCR3-activated Gαβγ dissociates into Gα and Gβγ, and because free Gβγ is capable of interacting with phospholipase Cβ to elevate the intracellular calcium ion concentration, it is also possible to conduct ligand screening, irrespective of the family of the coupling Gα, with the intracellular calcium ion as the index.

Here, “CXCR3” refers to CXCR3s derived from the human and other mammals, and a protein which comprises the amino acid sequence thereof with one or a plurality of amino acids substituted, deleted, inserted, added or modified, which exhibits the same ligand-receptor interaction as naturally-occurring CXCR3, and which possesses an activity that activates the coupling Gα to promote the GDP-GTP exchange reaction of said Gα.

CXCR3 can be isolated from a membrane-containing fraction of mammalian monocytes or Th1 cells by affinity chromatography using an anti-CXCR3 antibody. Alternatively, it is also possible to clone a DNA clone, which is isolated from a cDNA library or genomic library derived from such cells with a cDNA clone of CXCR3 as a probe, into an appropriate expression vector, transfect the vector to the host cell to express CXCR3, and purify it from a membrane-containing fraction of the cell culture by affinity chromatography using an anti-CXCR3 antibody, His-tag, GST-tag and the like. Also, CXCR3 may partially have a mutation transfected by an artificial treatment such as site-directed mutagenesis, based on a cDNA clone of CXCR3. However, because the ligand-binding domain needs to be highly conserved, it is desirable that no mutation be transfected to such regions. Conservative amino acid substitution is widely known; those skilled in the art can appropriately transfect a mutation to CXCR3, as long as the characteristics of CXCR3 are not altered.

Although the origin of the CXCR3-retaining lipid bilayer membrane is not subject to limitation, as long as the receptor is allowed to take its native steric structure; preferably, a fraction containing the cell membrane of a mammalian cell such as of the human, bovine, swine, monkey, mouse or rat, for example, an intact cell, a cell homogenate, or a cell membrane fraction fractionated from said homogenate by centrifugation and the like, can be mentioned. However, for example, an artificial lipid bilayer membrane prepared from a solution wherein various lipids such as phosphatidylcholine, phosphatidylserine and cholesterol are mixed in an appropriate ratio, preferably at a ratio close to that in the cell membrane of a mammalian cell, by a conventional method, can also be preferably used in a mode of embodiment of the present invention.

The Gα coupled with CXCR3 needs to have at least a region involved in the binding of GPCR to said Gα and a region involved in the binding to guanine nucleotide of an optionally chosen Gα. For example, when the Gα that is coupled with CXCR3 belongs to the Gi family (Giα), the Gα used has at least the GPCR-binding region of Giα, and also has the guanine nucleotide-binding region of Giα or the guanine nucleotide-binding region of a Gα belonging to another family. From the results of X-ray crystallographic analysis of Gα and the like, it has been evident that a sequence of about 5 amino acids or so at the C-terminus is important to binding to GPCR, and that the guanine nucleotide-binding region is a region homologous to the nucleotide-binding site of the ras protein (from the N-terminus side, amino acid motifs called P box, G′ box, G box, and G″ box, and the head of the αE helix and the αF helix in a highly-helixed domain, and the like).

Upon binding of a physiological ligand or agonist for CXCR3 binds to said receptor, the Gα activating domain of said receptor and the GPCR-binding region of Gα interact with each other to cause a conformational change of Gα, resulting in the dissociation of GDP from the guanine nucleotide-binding region and the quick binding of GTP. On the other hand, upon the binding of an inverse agonist, the activated type, Gα-GTP level decreases as the Gα activating domain is inactivated due to a conformational change of the receptor. Here, provided that a GTP analogue not undergoing hydrolysis by the GTPase activity of Gα, such as ³⁵S-labeled GTPγS, instead of GTP, has been added to the system, agonists or inverse agonists for CXCR3 can be screened by measuring and comparing the radioactivity bound to the membrane in the presence and absence of a test substance. That is, if the radioactivity increases in the presence of the test substance, said test substance is an agonist, and if the radioactivity decreases, said test substance is an inverse agonist.

Alternatively, screening can also be achieved by monitoring the binding of a GTP analogue to Gα using the surface plasmon resonance method and the like.

The activity of CXCR3 ligand can also be measured based on the action of the coupling Gα on the effector as the index. In this case, the screening system of the present invention needs to further contain, as a constituent, a lipid bilayer membrane containing an effector in addition to CXCR3. Also, the coupling Gα needs to further contain a region for interacting with said effector. The region may be the native effector interacting region of the Gα, and may be the effector interacting region of a Gα belonging to a different family. For example, with respect to Giα, as the Gα belonging to a different family, Gqα, Gsα, G12α and the like can be mentioned; with respect to Gqα, as the Gα belonging to a different family, Giα, Gsα, G12α and the like can be mentioned. As the simplest example of a chimeric Gα (for example, Ggα) containing the effector interacting region of the Gα belonging to a different family (for example, Giα), a Gα (Gqiα) wherein about 5 amino acids or so at the C-terminus of Gqα have been substituted by a C-terminus sequence of Giα can be mentioned.

When the Gα that is coupled with CXCR3 contains the effector interacting region of Giα, a lipid bilayer membrane containing adenylate cyclase as the effector is used. On the other hand, when the coupling Gα contains the effector interacting region of Gqα, a lipid bilayer membrane containing phospholipase Cβ as the effector needs to be used. Note that when the coupling Gα contains the effector interacting region of Gsα, a lipid bilayer membrane containing adenylate cyclase as the effector is used, and, in contrast to the case of Giα, ligand activity is evaluated with adenylate cyclase activity promoting action as the index.

In a screening system containing adenylate cyclase (hereinafter also referred to as AC) as the effector, the action of Gα on the effector can be evaluated by directly measuring AC activity. For the measurement of AC activity, any known technique may be used; for example, a method wherein ATP is added to an AC-containing membrane fraction, and the amount of cAMP produced is measured by competitive immunoassay with cAMP labeled with RI (¹²⁵I), an enzyme (alkaline phosphatase, peroxidase and the like), a fluorescent substance (FITC, rhodamine and the like) and the like using an anti-cAMP antibody, and a method wherein [α-³²P]ATP is added to an AC-containing membrane fraction, and the resulting [³²P]cAMP is separated using an alumina column and the like, after which the radioactivity is measured, can be mentioned, which methods, however, are not to be construed as limiting. Therefore, for example, when the Gα that coupling with CXCR3 contains the effector interacting region of Giα, and AC activity is measured and compared in the presence and absence of a test substance, if AC activity increases in the presence of the test substance, said test substance is an inverse agonist for CXCR3, and if the activity decreases, said test substance is an agonist.

When an intact eukaryotic cell is used as the screening system, the action of Gα on AC can also be evaluated by measuring the amount of cAMP in the cell, or by labeling the cell with [³H] adenine and measuring the radioactivity of the resulting [³H]cAMP. Although the amount of intracellular cAMP can be measured by incubating the cell in the presence and absence of a test substance for an appropriate time, then disrupting the cell, and subjecting the obtained extract to the above-described competitive immunoassay, any other known method can also be used.

In another embodiment, there is a method of evaluating the amount of cAMP by measuring the expression level of reporter gene under the control of the cAMP-responsive element (CRE). The expression vector used here is described in detail below; in summary, the amount of intracellular cAMP is evaluated by culturing an animal cell incorporating a vector containing an expression cassette wherein a DNA that encodes a reporter protein is ligated downstream of a CRE-containing promoter, in the presence and absence of a test substance for an appropriate time, disrupting the cell, and measuring and comparing the expression of the reporter gene in the obtained extract using a known technique.

Accordingly, for example, when the Gα that is coupled with CXCR3 contains the effector interacting region of Giα, if the amount of intracellular cAMP (or the expression level of reporter gene under the control of CRE) increases in the presence of a test substance, said test substance is an inverse agonist for CXCR3, and if the amount decreases, said test substance is an agonist.

On the other hand, in a screening system containing phospholipase Cβ (hereinafter also referred to as PLCβ) as the effector (that is, in cases where the Gα is Gqα or a chimeric protein containing the effector interacting region of Gqα (chimeric Gqα)), the action of said Gqα or chimeric Gqα on the effector can be evaluated by directly measuring PLCβ activity. PLCβ activity can, for example, be evaluated by adding ³H-labeled phosphatidylinositol-4,5-diphosphate to a PLCβ-containing membrane fraction, and measuring the amount of inositol phosphate produced using a known technique. When PLCβ activity is measured and compared in the presence and absence of a test substance, if PLCβ activity increases in the presence of the test substance, said test substance is an agonist for CXCR3, and if the activity decreases, said test substance is an inverse agonist.

When an intact eukaryotic cell is used as the screening system, the action of Gqα or chimeric Gqα on PLCβ can also be evaluated by adding [³H]inositol to the cell, and measuring the radioactivity of the [³H]inositol phosphate produced, or by measuring the amount of Ca²⁺ in the cell. Although the amount of intracellular Ca²⁺ can be measured spectrometrically using a fluorescent probe (fura-2, indo-1, fluo-3, Calcium-Green I and the like) after incubating the cell in the presence and absence of the test substance for an appropriate time, or can be measured using aequorin, which is a calcium-sensitive luminescent protein, and the like, any other known method may be used. As an apparatus suitable for the spectrometric measurement using a fluorescent probe, the FLIPR® (Molecular Devices) system can be mentioned.

In another embodiment, there is also a method of evaluating the amount of Ca²⁺ by measuring the expression level of reporter gene under the control of the TPA (12-O-tetradecanoylphorbol-13-acetate)-responsive element (TRE), which is upregulated by Ca²⁺. The expression vector used here is described in detail below; in summary, by culturing a eukaryotic cell incorporating a vector containing an expression cassette wherein a DNA that encodes a reporter protein is ligated downstream of a TRE-containing promoter, in the presence and absence of a test substance for an appropriate time, disrupting the cell, and measuring and comparing the expression of the reporter gene in the obtained extract using a known technique, the amount of intracellular Ca²⁺ is evaluated.

Therefore, if the amount of intracellular Ca²⁺ (or the expression level of reporter gene under the control of TRE) increases in the presence of the test substance, said test substance is an agonist for CXCR3, and if the amount decreases, said test substance is an inverse agonist.

Provided that the above-described screening method for CXCR3 ligand using CXCR3 and a coupling Gα is conducted in the co-presence of a known ligand for CXCR3, for example, IP-10, Mig, I-TAC, BCA-1 and the like, a neutral antagonist for CXCR3 can easily be selected.

The test substance subjected to the screening method of the present invention may be any known compound or novel compound; for example, a compound library prepared using combinatorial chemistry technology, a random peptide library prepared by solid phase synthesis or the phage display method, or naturally-occurring ingredients derived from microorganisms, animals, plants, marine organisms and the like, and the like can be mentioned.

A preferred embodiment of the screening system containing a CXCR3-containing lipid bilayer membrane and a Gα that is coupled with CXCR3 as the constituents, provided for the screening method of the present invention, is a host eukaryotic cell transfected with an expression vector containing a DNA that encodes CXCR3 and with an expression vector containing a DNA that encodes a polypeptide containing at least a region involved in the binding to GPCR of the coupling Gα and a region involved in the binding to guanine nucleotide of an optionally chosen Gα, a homogenate of said cell or a membrane fraction derived from said cell.

A “DNA that encodes CXCR3” is not subject to limitation, as long as it is a DNA that encodes a CXCR3 derived from the human or another mammal, or a polypeptide which comprises the amino acid sequence of said receptor with one or a plurality of amino acids substituted, deleted, inserted, added or modified, which exhibits the same ligand-receptor interaction as naturally-occurring CXCR3, and which possesses an activity that activates the coupling Gα to promote the GDP-GTP exchange reaction of said subunit. In addition to the coding region of human CXCR3 cDNA, DNAs that encode CXCR3s derived from non-human mammals such as the bovine, swine, monkey, mouse and rat, and the like can be mentioned as examples; these can be isolated from a cDNA library or genomic library derived from mammalian monocytes or Th1 cells, with a cDNA clone of human CXCR3 as the probe. Also, CXCR3 may partially have a mutation transfected by an artificial treatment such as site-directed mutagenesis, based on a cDNA clone of human CXCR3.

As the Gα, there is no limitation, as long as it is coupled with CXCR3. The respective genes of Gαs are known and easily available. The DNA that encodes a polypeptide containing a Gα that is coupled with CXCR3 needs to have at least the sequence that encodes the region involved in the binding to GPCR of the coupling Gα, and a sequence that encodes a region involved in the binding to guanine nucleotide of an optionally chosen Gα. As described above, from the results of X-ray crystallographic analysis of Gα, the GPCR-binding region and the guanine nucleotide-binding region are well known; those skilled in the art can easily construct a fragment lacking a portion of the coding sequence of Gα where desired.

In a screening system with the action of Gα on the effector as the index, the DNA that encodes the Gα that is coupled with CXCR3 needs to further contain the nucleotide sequence that encodes a region for interacting with the desired effector. When adenylate cyclase is used as the effector, said DNA contains the nucleotide sequence that encodes the effector interacting region of Giα or Gsα. On the other hand, when phospholipase Cβ is used as the effector, said DNA contains the nucleotide sequence that encodes the effector interacting region of Gqα. The respective Gα genes are known, and their effector interacting regions are also well known. Therefore, those skilled in the art can also easily construct a DNA that encodes a chimeric Gα protein by appropriately combining known gene engineering techniques. As the simplest example of the DNA that encodes a chimeric protein (for example, Gqiα), the sequence that encodes about 5 amino acids at the C-terminus of Gqα cDNA, substituted by the DNA sequence that encodes a C-terminus sequence of Giα, using a known technique such as PCR, can be mentioned.

The DNA that encodes CXCR3 and the DNA that encodes the Gα that is coupled with CXCR3 must be operably linked to a promoter capable of exhibiting promoter activity in the host eukaryotic cell. The promoter used is not subject to limitation, as long as it is capable of functioning in the host eukaryotic cell; for example, viral promoters such as the SV40-derived early promoter, cytomegalovirus LTR, Rous sarcoma virus LTR, MoMuLV-derived LTR, the adenovirus-derived early promoter and baculovirus-derived polyhedrin promoter; promoters of constitutive protein genes in eukaryote-derived cell such as the β-actin gene promoter, PGK gene promoter and transferrin gene promoter; and the like can be mentioned. The expression vector used preferably contains, in addition to the above-described promoter, a transcription termination signal, that is, a terminator region, downstream thereof, and desirably has an appropriate restriction endonuclease recognition site, preferably a unique restriction endonuclease recognition site to cleave said vector only at one site, so that a coding DNA can be inserted between the promoter region and the terminator region. Furthermore, said expression vector may further contain a selection marker gene (a drug resistance gene such as for tetracycline, ampicillin, kanamycin, hygromycin or phosphinothricin, an auxotrophic mutation complementary genes and the like).

As the vector used in the screening system of the present invention, in addition to plasmid vectors, retrovirus, adenovirus, adeno-associated virus, herpes virus, vaccinia virus, pox virus, polio virus, Sindbis virus, Sendai virus and the like, which are suitable for use in mammalians such as the human, or baculovirus vectors and the like, which are suitable for use in insect cells, can also be mentioned.

The DNA that encodes CXCR3, and the DNA that encodes the Gα that is coupled with CXCR3 may be carried on two separate expression vectors and co-transfected to the host cell, or may be di-cistronically or mono-cistronically inserted into a single vector and transfected into the host cell.

The host cell is not subject to limitation, as long as it is a mammalian cell such as of the human, monkey, mouse, rat or hamster, or an insect cell. Specifically, mouse-derived cells such as COP, L, C127, Sp2/0, NS-1, NIH3T3 and ST2, rat-derived cells, hamster-derived cells such as BHK and CHO, monkey-derived cells such as COS1, COS3, COS7, CV1 and Vero, human-derived cells such as HeLa and 293, insect-derived cells such as Sf9, Sf21 and High Five, and the like can be mentioned as examples.

Gene transfection to the host cell may be conducted using any known method usable for gene transfection to eukaryotic cells; for example, the calcium phosphate co-precipitation method, the electroporation method, the liposome method, the microinjection method and the like can be mentioned.

The gene-incorporating host cell can, for example, be cultured using a minimum essential medium (MEM) containing about 5˜20% of fetal bovine serum, Dulbecco's modified Eagle medium (DMEM), Ham's F-12 medium, RPMI1640 medium, 199 medium, Grace's insect cell culture medium and the like. It is preferable that the pH of the medium be about 6 to about 8; culturing temperature is normally about 27 to about 40° C.

The eukaryotic cell incorporating a DNA that encodes CXCR3 and a DNA that encodes the Gα that is coupled with CXCR3, obtained as described above, may be used as an intact cell as is, or may be in the form of a cell homogenate obtained by disrupting said cell in an appropriate buffer solution or a membrane fraction isolated by centrifuging said homogenate under appropriate conditions and the like (for example, after centrifugation at about 1,000×g or so and recovery of the supernatant, centrifugation is conducted at about 100,000×g or so and the sediment is recovered), depending on the screening method used.

For example, when the ligand characteristic of the test substance is evaluated by GTPγS binding assay or by directly measuring the activity of the effector, the screening system used is preferably a membrane fraction prepared from the cell as described above. On the other hand, when the ligand characteristic of the test substance is evaluated by measuring the amount of intracellular cAMP (or the expression level of cAMP-responsive reporter) or the amount of intracellular Ca²⁺ (or the expression level of Ca²⁺-responsive reporter), the screening system used is an intact eukaryotic cell.

Note that when the evaluation of ligand activity is conducted with the expression level of cAMP-responsive reporter (in cases where the effector is adenylate cyclase) or Ca²⁺-responsive reporter (in cases where the effector is phospholipase Cβ) as the index, the host eukaryotic cell needs to incorporate a vector containing an expression cassette wherein a DNA that encodes a reporter protein is operably linked downstream of a promoter region containing the cAMP-responsive element (CRE) or the TPA-responsive element (TRE). CRE is a cis-element that activates gene transcription in the presence of cAMP; although a sequence containing TGACGTCA as the consensus sequence can be mentioned, the consensus sequence may be a sequence containing a deletion, substitution, insertion or addition in a portion of the above-mentioned sequence, as long as cAMP responsiveness is retained. On the other hand, TRE is a cis-element that activates gene transcription in the presence of Ca²⁺; although a sequence containing TGACTCA as the consensus sequence can be mentioned, the consensus sequence may be a sequence containing a deletion, substitution, insertion or addition in a portion of the sequence, as long as Ca²⁺ responsiveness is retained. As the promoter sequence containing CRE or TRE, viral promoters and mammalian constitutive protein gene promoters as described above can be used in the same manner; using restriction endonuclease and DNA ligase, or utilizing PCR and the like, a CRE or TRE sequence can be inserted downstream of said promoter sequence. As the reporter gene under the control of CRE or TRE, any known gene permitting the quick and simple detection and quantitation of gene expression may be used; for example, DNAs that encode reporter proteins such as luciferase, β-galactosidase, β-glucuronidase, alkaline phosphatase and peroxidase can be mentioned, which, however, are not to be construed as limiting. It is more preferable that a terminator sequence be arranged downstream of the reporter gene. As the vector carrying such a CRE (or TRE)-reporter expression cassette, known plasmid vector or viral vector can be used.

Another preferred mode of embodiment of the screening system containing a CXCR3-containing lipid bilayer membrane and a Gα that is coupled with CXCR3 as the constituents, provided for the screening method of the present invention, is a host eukaryotic cell transfected with an expression vector containing a DNA that encodes a fusion protein wherein a polypeptide containing at least the GPCR-binding region of the coupling Gα and a guanine nucleotide-binding region of an optionally chosen Gα is ligated to the C-terminus side of CXCR3, a homogenate of said cell or a membrane fraction derived from said cell.

A DNA that encodes CXCR3 and a DNA that encodes a polypeptide containing the GPCR-binding region of a Gα that is coupled with CXCR3 and a guanine nucleotide-binding region of an optionally chosen Gα can be obtained as described above. Those skilled in the art can construct a DNA that encodes a fusion protein of CXCR3 and Gα, on the basis of these DNA sequences, by appropriately combining known gene engineering techniques. Briefly speaking, to a DNA that encodes CXCR3 wherein its stop codon has been removed using PCR and the like, a DNA that encodes Gα is ligated, so that the reading frames match, i.e., in-frame, using DNA ligase. At the time, the C-terminus of CXCR3 may be partially deleted, and a linker sequence such as the His tag may be inserted between CXCR3 and Gα.

The obtained DNA encoding the fusion protein is inserted to an expression vector as described above, and transfected to a host eukaryotic cell using the above-described gene transfection technology. Upon the expression of the fusion protein on the membrane of the obtained eukaryotic cell, the Gα-activating domain on the intracellular loop 3 of the receptor and the receptor-binding region of the coupling Gα can interact with each other in the absence of a physiological ligand for CXCR3 to promote the GDP-GTP exchange reaction in Gα. Therefore, Gα comes into a constitutively activated state.

In cases where a receptor-Gα fusion protein expressing cell is used for screening utilizing the action of Gα on the effector as the index, if the ligation of Gα to the receptor interferes with the interaction with the effector, it is possible to transfect, to the junction site of the receptor and Gα, an amino acid sequence cleaved with a specific protease (for example, thrombin-sensitive sequence and the like), express the fusion protein on the membrane, then allow the protease to act to cut away the receptor and Gα.

For the receptor-Gα fusion protein expression cell as well, any form of an intact cell, cell homogenate and membrane fraction, depending on the screening method used, can be appropriately selected and used.

Still another mode of embodiment of the screening system containing a CXCR3-containing lipid bilayer membrane and a Gα that is coupled with CXCR3 as the constituents, provided for the screening method of the present invention, is a cell prepared by transfecting a host animal cell that endogenously expresses the coupling G protein with an expression vector containing a DNA that encodes CXCR3, a homogenate of said cell or a membrane fraction derived from said cell. For the DNA that encodes CXCR3, the expression vector for insertion of said DNA, and the method of transfecting said expression vector to the host cell, the same as those described above can be used.

In still another embodiment of the present invention, the screening system of the present invention is an animal cell that endogenously expresses CXCR3 and a G protein that is coupled with CXCR3, a homogenate of said cell or a membrane fraction derived from said cell. As preferable examples of such cells, mammalian monocytes or Th1 cells can be mentioned.

In still another embodiment of the present invention, as the screening system containing a CXCR3-containing lipid bilayer membrane and a Gα that is coupled with CXCR3 as the constituents, purified CXCR3 and coupling Gα, or a purified fusion protein of said receptor and coupling Gα, reconstituted in an artificial lipid bilayer membrane, can be used. CXCR3 can be purified from a membrane fraction obtained from a monocyte or Th1 cell of the human or another mammalian animal, by affinity chromatography using an anti-CXCR3 antibody, and the like. Alternatively, said receptor can also be purified from a recombinant cell incorporating an expression vector containing a DNA that encodes CXCR3, by affinity chromatography using an anti-CXCR3 antibody, His-tag, GST-tag and the like, and the like. Likewise, a fusion protein of said receptor and coupling Gα can also be purified from a recombinant cell incorporating an expression vector containing a DNA that encodes said fusion protein, by affinity chromatography using an anti-CXCR3 antibody, His-tag, GST-tag and the like, and the like.

As a lipid composing an artificial lipid bilayer membrane, phosphatidyl choline (PC), phosphatidyl serine (PS), cholesterol (Ch), phosphatidyl inositol (PI), phosphatidyl ethanolamine (PE) and the like can be mentioned. A mixture of one or more kinds thereof mixed at a suitable ratio is preferably used.

For example, an artificial lipid bilayer membrane (proteoliposome) incorporating a receptor and Gα or a receptor-Gα fused protein can be prepared by the following methods. First, a suitable amount of a mixed lipid chloroform solution of PC:PS:Ch=12:12:1 is separated in a glass tube, chloroform is evaporated in a nitrogen gas vapor to dry the lipid in the form of a film, a suitable buffer is added to suspend the lipid, which is uniformly dispersed by ultrasonication, a buffer containing a surfactant such as sodium cholate and the like is further added to completely suspend the lipid. Thereto is added a suitable amount of purified receptor and Gα, or a receptor-Gα fused protein, and after incubation for about 20-30 min while sometimes stirring in ice water, dialyzed against a suitable buffer, centrifuged at about 100,000×g for 30-60 min and the sediment is recovered to give a desired proteoliposome.

It is evident that the CXCR3 agonists selected by the above-described screening method of the present invention, like the physiological ligand for CXCR3, exhibit impaired glucose tolerance ameliorating action; therefore, by combining them with an appropriate additive, they can be made impaired glucose tolerance ameliorating drugs. Accordingly, the present invention also provides an impaired glucose tolerance ameliorating drug or therapeutic drug for lifestyle-related diseases, particularly therapeutic drug for diabetes, which is formulated with a CXCR3 agonist selected by the screening method of the present invention as an active ingredient, and with a pharmaceutically acceptable carrier added where necessary.

Also, because the CXCR3 antagonist or inverse agonist selected by the above-described screening method of the present invention is assumed to reduce insulin secretion when administered, it is applicable to various diseases against which it is considered to have an effect due to a reduction in insulin secretion. Also, when the CXCR3 antagonist is a substance for food use that has conventionally been safely taken by animals, such substances are also applicable to uses such as diet foods.

The pharmaceutically acceptable carrier is exemplified by, but not limited to, excipients such as sucrose, starch, mannit, sorbit, lactose, glucose, cellulose, talc, calcium phosphate, calcium carbonate and the like, binders such as cellulose, methylcellulose, hydroxypropylcellulose, polypropylpyrrolidone, gelatine, gum arabic, polyethylene glycol, sucrose, starch and the like, disintegrating agents such as starch, carboxymethyl cellulose, hydroxypropyl starch, sodium-glycol-starch, sodium hydrogen carbonate, calcium phosphate, calcium citrate and the like, lubricants such as magnesium stearate, aerosil, talc, sodium lauryl sulfate and the like, aromatics such as citric acid, menthol, glycyl lysine ammonium salt, glycine, orange powder and the like, preservatives such as sodium benzoate, sodium bisulfite, methylparaben, propylparaben and the like, stabilizers such as citric acid, sodium citrate, acetic acid and the like, suspending agents such as methylcellulose, polyvinylpyrrolidone, aluminum stearate and the like, dispersing agents such as surfactant and the like, diluents such as water, physiological saline, orange juice and the like, base wax such as cacao butter, polyethylene glycol, refined kerosene and the like, and the like.

Preparations suitable for oral administration are a liquid comprising an effective amount of CXCR3 ligand dissolved in a diluent like water, physiological saline or orange juice, a capsule, sachet or tablet containing an effective amount of CXCR3 ligand as a solid or granules, a suspension comprising an effective amount of CXCR3 ligand suspended in an appropriate dispersion medium, an emulsion comprising a solution of an effective amount of CXCR3 ligand dispersed and emulsified in an appropriate dispersion medium, and the like.

As preparations suitable for parenteral administration (for example, intravenous injection, intra-arterial injection, subcutaneous injection, intramuscular injection, topical injection, intraperitoneal administration and the like), aqueous and non-aqueous isotonic sterile injectable liquids are available, which may contain an antioxidant, a buffer solution, a bacteriostatic agent, an isotonizing agent and the like. Also, aqueous and non-aqueous sterile suspensions can be mentioned, which may contain a suspending agent, a solubilizer, a thickener, a stabilizer, an antiseptic and the like. Alternatively, a sustained-release preparation may be prepared using a biocompatible material such as collagen. The CXCR3 ligand preparation can be sealed in a container like ampoules and vials for a unit dose or multiple doses. Also, the CXCR3 ligand and pharmaceutically acceptable carrier can also be lyophilized and preserved in a state that only requires dissolving or suspending in an appropriate sterile vehicle just before use.

Although the dose and administration frequency of the CXCR3 ligand preparation vary depending on symptoms, age, body weight, dosage form and the like, the preparation can usually be administered in the range of about 0.0001 to about 500 mg, preferably in the range of about 0.001 to about 100 mg per day for each adult once or in several divided portions.

According to the present invention, it was found that in the blood of type II diabetes patients, IP-10, Mig, I-TAC or BCA-1 and the like, which are physiological ligands for CXCR3, are present at higher concentrations than those in healthy people. Accordingly, the present invention also provides a diagnostic method for type II diabetes comprising measuring a CXCR3 ligand or the transcript of the gene of said ligand in a biological sample using an antibody possessing specific affinity for the physiological ligand for CXCR3 or a DNA that encodes said ligand or a DNA hybridizable with said DNA under stringent conditions.

The anti-CXCR3 ligand antibody may be any of a polyclonal antibody and a monoclonal antibody, and can be prepared by a widely known immunological technique. The fragment of anti-CXCR3 ligand antibody may be any one, as long as it has an antigen-binding site (CDR) for a physiological ligand for CXCR3 such as IP-10, Mig, I-TAC and BCA-1; for example, Fab, F(ab′)2, ScFv, minibody and the like can be mentioned.

For example, the polyclonal antibody can be obtained by subcutaneously or intraperitoneally administering a physiological ligand for CXCR3 or a fragment thereof (where necessary, may be prepared as a complex crosslinked with a carrier protein such as bovine serum albumin or KLH (Keyhole Limpet Hemocyanin)) as the antigen, along with a commercially available adjuvant (for example, complete or incomplete Freund's adjuvant), to an animal at intervals of 2˜3 weeks 2˜4 times or so (the antibody titer in a serum of partially drawn blood has been measured by a known antigen-antibody reaction, and its elevation has been confirmed), collecting whole blood about 3 to about 10 days after final immunization, and purifying the anti-serum. As animals to receive the antigen, mammals such as the rat, mouse, rabbit, goat, guinea pig and hamster can be mentioned.

Also, the monoclonal antibody can be prepared by a cell fusion method (for example, Takeshi Watanabe, Saibou Yugouhou No Genri To Monokuronaru Koutai No Sakusei, edited by Akira Taniuchi and Toshitada Takahashi, “Monokuronaru Koutai To Gan—Kiso To Rinsho—”, pp. 2-14, Science Forum Shuppan, 1985). For example, said factor, along with a commercially available adjuvant, is subcutaneously or intraperitoneally administered to a mouse 2˜4 times, and about 3 days after final administration, the spleen or lymph node is collected and leukocytes are collected. These leukocytes and myeloma cells (for example, NS-1, P3X63Ag8 and the like) are subjected to cell fusion to yield a hybridoma that produces a monoclonal antibody against said factor. Cell fusion may be achieved by the PEG method [J. Immunol. Methods, 81(2): 223-228 (1985)] or the voltage pulse method [Hybridoma, 7(6): 627-633 (1988)]. A hybridoma that produces the desired monoclonal antibody can be selected by detecting an antibody that specifically binds to the antigen from the culture supernatant, using a widely known EIA or RIA method and the like. Cultivation of the hybridoma that produces the monoclonal antibody can be conducted in vitro, or in vivo such as in the ascites fluid of the mouse or rat, preferably the mouse, and the antibody can be obtained from the hybridoma culture supernatant and the animal ascites fluid, respectively.

A DNA that encodes a physiological ligand for CXCR3 or a DNA hybridizable with said DNA under stringent conditions can easily be obtained on the basis of the known cDNA sequences of human IP-10, Mig, I-TAC and BCA-1. Here, “stringent conditions” are conditions under which a DNA possessing about 80% or higher identity to the target DNA hybridizes; conditions under which a DNA preferably possessing about 90% or higher, more preferably about 95% or higher identity hybridizes, are selected. Such stringency can easily be regulated by appropriately selecting salt concentrations of a hybridization buffer and a washing solution, hybridization and washing temperatures and the like.

As examples of biological samples used in the diagnostic method of the present invention, blood, plasma, serum, urine, blood cell homogenates, tissues (for example, muscular tissue and adipose tissue) obtained by biopsy and the like can be mentioned, which, however, are not to be construed as limiting.

The present invention also provides an impaired glucose tolerance ameliorating drug and therapeutic drug for lifestyle-related diseases, particularly for diabetes, containing a substance that inhibits the expression or activity of CXCR3, other than CXCR3 agonists, as an active ingredient. As such substances, a CXCR3 protein or a modification thereof, or an expression vector containing a DNA that encodes them, and the like can be mentioned. As examples of the CXCR3 protein, a human-derived protein having the amino acid sequence shown by SEQ ID NO: 2 can be mentioned, and as the modification, a protein which has said amino acid sequence with one or a plurality of amino acids substituted, deleted, inserted, added or modified, and which retains the physiological activity of CXCR3 can be mentioned. An expression vector containing a DNA that encodes a CXCR3 protein or a modification thereof can be constructed by the same technique as the above-described expression vector containing a DNA that encodes a CXCR3 ligand.

The present invention also provides a hypoglycemia ameliorating drug, therapeutic drug for diseases that can be ameliorated by insulin secretion suppression, and anti-obesity drug containing a substance that inhibits the expression or activity of CXCR3, other than CXCR3 antagonists, as an active ingredient.

A preferred embodiment of the substance that inhibits the expression of CXCR3 is an antisense nucleic acid of the mRNA or primary transcript of CXCR3. “Antisense nucleic acid” refers to a nucleic acid which comprises a base sequence hybridizable with a target mRNA (primary transcript) under the physiological conditions for the cell that expresses the target mRNA (primary transcript), and which is capable of inhibiting the translation of the polypeptide encoded by said target mRNA (primary transcript) in the hybridized state. The kind of antisense nucleic acid may be DNA or RNA, or may be a DNA/RNA chimera.

The length of the antisense nucleic acid of the present invention is not subject to limitation, as long as it is specifically hybridizable with the mRNA or primary transcript of CXCR3, and the antisense nucleic acid may be a sequence containing a sequence of about 15 bases or so at shortest or complementary to the entire sequence of the mRNA (primary transcript) at longest. From the viewpoint of the ease of synthesis, antigenicity concern and the like, oligonucleotides consisting of about 15 to about 30 bases can be mentioned as preferable examples. When the antisense nucleic acid is an oligo-DNA of about 25mer, the base sequence hybridizable with the mRNA of CXCR3 under physiological conditions varies depending on the base composition of the target sequence, and may normally be any one, as long as it possesses about 80% or higher identity.

The antisense oligonucleotide of the present invention can be prepared by determining the target sequence of the mRNA or primary transcript on the basis of the cDNA sequence or genomic DNA sequence of CXCR3, and synthesizing a sequence complementary thereto using a commercially available DNA/RNA synthesizer (Applied Biosystems, Beckman and the like).

A preferred embodiment of the substance that inhibits the functional expression of CXCR3 at the post-transcriptional level is an antibody against CXCR3 or a fragment thereof. Said antibody may be any of a polyclonal antibody and a monoclonal antibody, and can be prepared by a widely known immunological technique. The fragment of the anti-CXCR3 antibody may be any one, as long as it has an antigen-binding site (CDR) for CXCR3; for example, Fab, F(ab′)₂, ScFv, minibody and the like can be mentioned.

Considering the therapeutic effect and safety in the human, the anti-CXCR3 antibody of the present invention is preferably a chimeric antibody of the human and another animal (for example, mouse and the like), more preferably a humanized antibody. Here, “chimeric antibody” refers to an antibody having a variable region (V region) derived from an immunized animal and a human-derived constant region (C region), and “humanized antibody” refers to an antibody replaced with a human antibody at all regions except CDR. A chimeric antibody and a humanized antibody can, for example, be obtained by cutting out the sequence that encodes the V region or CDR from the gene of a mouse monoclonal antibody prepared by the same method as described above, fusing the sequence with a DNA that encodes the C region of a human-myeloma-derived antibody, cloning the resulting chimeric gene into an appropriate expression vector, and transfecting this to an appropriate host cell to express said chimeric gene.

The present invention is hereinafter described in more concretely by means of the following examples, which examples, however, are given for the sake of exemplification and do not limit the scope of the present invention.

### REFERENCE EXAMPLE 1

### Localized Expression of CXCR3 in the Normal Human Pancreas

DNA chip analysis was conducted using total RNAs prepared from various normal human tissues (adipose, cerebellum, heart, hippocampus, kidney, liver, lung, muscle, pancreas, small intestine, spleen, stomach, testis, thymus, leukocytes). The DNA chip analysis was conducted using Affymetrix Gene Chip Human Genome U95A,B,C,D,E. Specifically, the analysis was conducted in the procedures of (1) preparation of cDNA from total RNA, (2) preparation of labeled cRNA from said cDNA, (3) fragmentation of labeled cRNA, (4) hybridization of fragmented cRNA and probe array, (5) staining of probe array, (6) scanning of probe array and (7) gene expression analysis.

(1) Preparation of cDNA from Total RNA

11 μL of a mixed liquid containing 10 μg of each total RNA prepared from each normal human tissue and 100 pmol of the. T7-(dT)24 primer (manufactured by Amersham) was heated at 70° C. for 10 minutes, after which it was cooled on ice. After cooling, 4 μL of 5× First Strand cDNA Buffer contained in the SuperScript Choice System for cDNA Synthesis (manufactured by Gibco-BRL), 2 μL of 0.1M DTT (dithiothreitol) included in said kit, and 1 μL of 10 mM dNTP Mix included in said kit were added, and heating was conducted at 42° C. for 2 minutes. Further, 2 μL (400 U) of Super ScriptII RT included in said kit was added, and heating was conducted at 42° C. for 1 hour, after which the mixture was cooled on ice. After cooling, 91 μL of DEPC-treated water (manufactured by Nacalai Tesque), 30 μL of 5× Second Strand Reaction Buffer included in said kit, 3 μL of 10 mM dNTP Mix, 1 μL (10 U) of E. coli DNA Ligase included in said kit, 4 μL (40 U) of E. coli DNA Polymerase I included in said kit, and 1 μL (2 U) of E. coli RNaseH included in said kit were added, and the mixture was reacted at 16° C. for 2 hours. Next, 2 μL (10U) of T4 DNA Polymerase included in said kit was added, and the mixture was reacted at 16° C. for 5 minutes, after which 10 μL of 0.5M EDTA was added. Next, 162 μL of a phenol/chloroform/isoamyl alcohol solution (manufactured by Nippon Gene) was added and mixed. Said mixed liquid was transferred to Phase Lock Gel Light (manufactured by Eppendorf), previously centrifuged at room temperature and 14,000 rpm for 30 seconds, and centrifuged at room temperature and 14,000 rpm for 2 minutes, after which 145 μL of the water layer was transferred to an Eppendorf tube. To the obtained solution, 72.5 μL of a 7.5M ammonium acetate solution and 362.5 μL of ethanol were added and mixed, after which centrifugation was conducted at 4° C. and 14,000 rpm for 20 minutes. After centrifugation, the supernatant was discarded to yield DNA pellet containing the prepared cDNA. Subsequently, 0.5 mL of 80% ethanol was added to said pellet and centrifugation was conducted at 4° C. and 14,000 rpm for 5 minutes, after which the supernatant was discarded. After the same operation was again conducted, said pellet was dried and dissolved in 12 μL of DEPC-treated water. Through the procedures above, cDNA was obtained from total RNA.

(2) Preparation of Labeled cRNA from cDNA

To 5 μL of each cDNA solution prepared in (1) above, 17 μL of DEPC-treated water, 4 μL of 10× HY Reaction Buffer contained in the BioArray High Yield RNA Transcript Labeling Kit (manufactured by ENZO), 4 μL of 10× Biotin Labeled Ribonucleotides included in said kit, 4 μL of 10× DTT included in said kit, 4 μL of 10× RNase Inhibitor Mix included in said kit, and 2 μL of 20× T7 RNA Polymerase included in said kit were mixed, and the mixture was reacted at 37° C. for 5 hours. After the reaction, 60 μL of DEPC-treated water was added to said reaction liquor, after which the prepared labeled cRNA was purified using RNeasy Mini Kit per the attached protocol.

(3) Fragmentation of Labeled cRNA

To a solution containing 20 μg of each labeled cRNA purified in (2) above, 8 μL of 5× Fragmentation Buffer (200 mM Tris-acetic acid, pH 8.1 (manufactured by Sigma), 500 mM potassium acetate (manufactured by Sigma) and 150 mM magnesium acetate (manufactured by Sigma)) was added; 40 μL of the obtained reaction liquor was heated at 94° C. for 35 minutes, after which it was placed in ice. The labeled cRNA was thereby fragmented.

(4) Hybridization of Fragmented cRNA and Probe Array

To 40 μL of each fragmented cRNA obtained in (3) above, 4 μL of 5 nM Control Oligo B2 (manufactured by Amersham), 4 μL of 100× Control cRNA Cocktail, 40 μg of Herring sperm DNA (manufactured by Promega), 200 μg of Acetylated BSA (manufactured by Gibco-BRL), 200 μL of 2× MES Hybridization Buffer (200 mM MES, 2M [Na+], 40 mM EDTA, 0.02% Tween 20 (manufactured by Pierce), pH 6.5-6.7) and 144 μL of DEPC-treated water were mixed, to yield 400 μL of a hybridization cocktail. Each obtained hybridization cocktail was heated at 99° C. for 5 minutes and further heated at 45° C. for 5 minutes. After heating, centrifugation was conducted at room temperature and 14,000 rpm for 5 minutes to yield a hybridization cocktail supernatant.

On the other hand, a Human genome U95 probe array (manufactured by Affymetrix) filled with the 1× MES hybridization buffer was rotated in a hybridization oven at 45° C. and 60 rpm for 10 minutes, after which the 1× MES hybridization buffer was removed to prepare the probe array. 200 μL of the hybridization cocktail supernatant obtained above was each added to said probe array, and the array was rotated in a hybridization oven at 45° C. and 60 rpm for 16 hours, to yield a probe array hybridized with the fragmented cRNA.

(5) Staining of Probe Array

From each of the hybridized probe arrays obtained in (4) above, the hybridization cocktail was recovered and removed, after which the array was filled with Non-Stringent Wash Buffer (6× SSPE (20× SSPE (manufactured by Nacalai Tesque) was diluted), 0.01% Tween 20 and 0.005% Antifoam 0-30 (manufactured by Sigma)). Next, to the preset position of GeneChip Fluidics Station 400 (manufactured by Affymetrix) with Non-Stringent Wash Buffer and Stringent Wash Buffer (100 mM MES, 0.1 M NaCl and 0.01% Tween 20) set thereto, the probe array that hybridized with the fragmented cRNA was placed. Subsequently, per the staining protocol EuKGE-WS2, the probe array was stained with a primary staining solution (10 μg/mL Streptavidin Phycoerythrin (SAPE) (manufactured by Molecular Probe), 2 mg/mL Acetylated BSA, 100 mM MES, 1 M NaCl (manufactured by Ambion), 0.05% Tween 20 and 0.005% Antifoam 0-30), and a secondary staining solution (100 μg/mL Goat IgG (manufactured by Sigma), 3 μg/mL Biotinylated Anti-Streptavidin antibody (manufactured by Vector Laboratories), 2 mg/mL Acetylated BSA, 100 mM MES, 1 M NaCl, 0.05% Tween 20 and 0.005% Antifoam 0-30), respectively.

(6) Scanning of Probe Array and (7) Gene Expression Analysis

Each probe array stained in (5) above was applied to the HP GeneArray Scanner (manufactured by Affymetrix), and the staining pattern was read. Based on the staining pattern, gene expression on the probe array was analyzed using the GeneChip Workstation System (manufactured by Affymetrix). Next, per the analytical protocol, Normalization and comparative analysis of gene expression was conducted.

As a result, it became evident that CXCR3 was specifically expressed in the pancreas compared to other tissues (the relative expression values in the other tissues were −23-72, whereas that in the pancreas was 668; see Table 1) .

TABLE 1
Tissue distribution of CXCR3 gene expression

| fat | cerebellum | heart | hippocampus | kidney |
|---|---|---|---|---|
| 41 | 46 | −22 | 33 | 53 |
| liver | lung | muscle | pancreas | small |
| | | | | intestine |
| 32 | 33 | 58 | 668 | 70 |
| spleen | stomach | testis | thymus | leukocyte |
| 52 | 54 | 37 | 60 | 72 |

### REFERENCE EXAMPLE 2

### Localized Expression of CXCR3 in the Pancreatic Islet

The expression of CXCR3 in the pancreatic islet was examined for by the Western blot method.

The pancreatic islet was isolated from an ICR mice (Clea Japan) using a method described in the literature (The Journal of Physiology, 521 (3), 717-728 (1999)). After one day of cultivation, cells were harvested via centrifugation and washed with PBS several times, after which they were dissolved with 0.25 ml of RIPA buffer (1% NP40, 20 mM Tris-HCl pH 7.4, 150 mM NaCl, 0.1% Sodium deoxycholate, 5 mM EDTA, 10 mM NaF, 2 mM Na₃VO₄, 10 μg/ml leupepsin, 10 μg/ml aprotinin, 1 mM PMSF) by pipetting. Also, the pancreas was separately extirpated and disrupted, with the addition of 1 ml of RIPA buffer, using a homogenizer. The insoluble matter of these extracts was removed via centrifugation, an equal amount of 2× SDS sample Buffer was added to the supernatant, and the supernatant was boiled, after which the supernatant was subjected to SDS-PAGE with Multigel10/20 (Daiichi Pure Chemicals) and transferred to a PVDF filter (Amersham Life Science), after which blocking was conducted for 1 hour using a blocking buffer (1×TBS, 0.1% Tween 20, 5% Skim milk). A primary antibody reaction was conducted using an anti-CXCR3 antibody (SNATA CRUZ Cat. No. sc-13951: 250 fold diluted with PBST). A HRP-conjugated anti-rabbit antibody (Amersham Life Science: 1000 fold diluted with PBST) was used as the secondary antibody. Detection was conducted using the ECL-Plus detection system (Amersham Life Science).

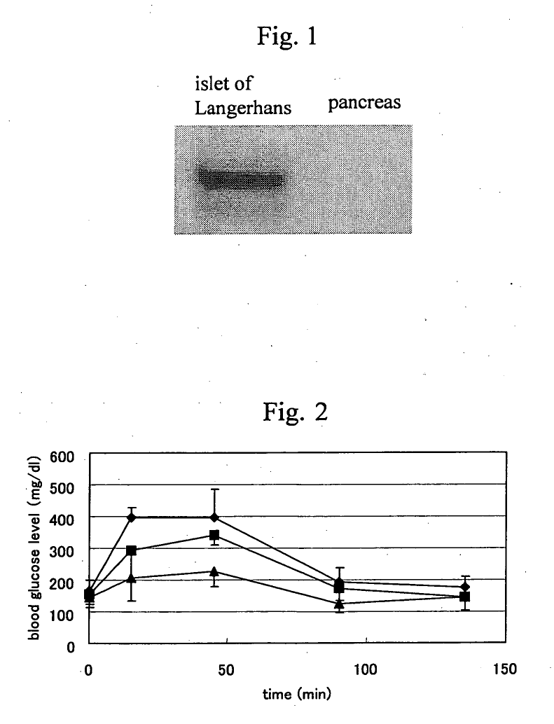

As a result, it became evident that CXCR3 was locally expressed in the pancreatic islet (FIG. 1). From this result, it was suggested that CXCR3 might be involved in regulation of insulin secretion.

### EXAMPLE 1

### Oral Glucose Tolerance Test

In the test, diabetic model mouse C57BL/KsJ-db/db mice (male, SPF grade, 7-week old, Clea Japan) were fasted overnight from the day before testing, mouse IP-10 (PEPRO TECH) or GLP-1 (7-36 Amide) (Peptide Institute), previously dried, was dissolved in physiological saline (Otsuka Pharmaceutical), and intravenous administration was conducted (6 μg/head; n=4 for IP-10, 50 μg/head; n=4 for GLP-1). For a solvent control group, physiological saline (Otsuka Pharmaceutical) was used (n=4). After intravenous administration, 3 g/kg of D-glucose was orally administered. Before glucose loading (0 minute) and 15, 45, 90 and 135 minutes after glucose loading, blood was collected from the caudal vein, and blood glucose levels were measured in accordance with the method described below.

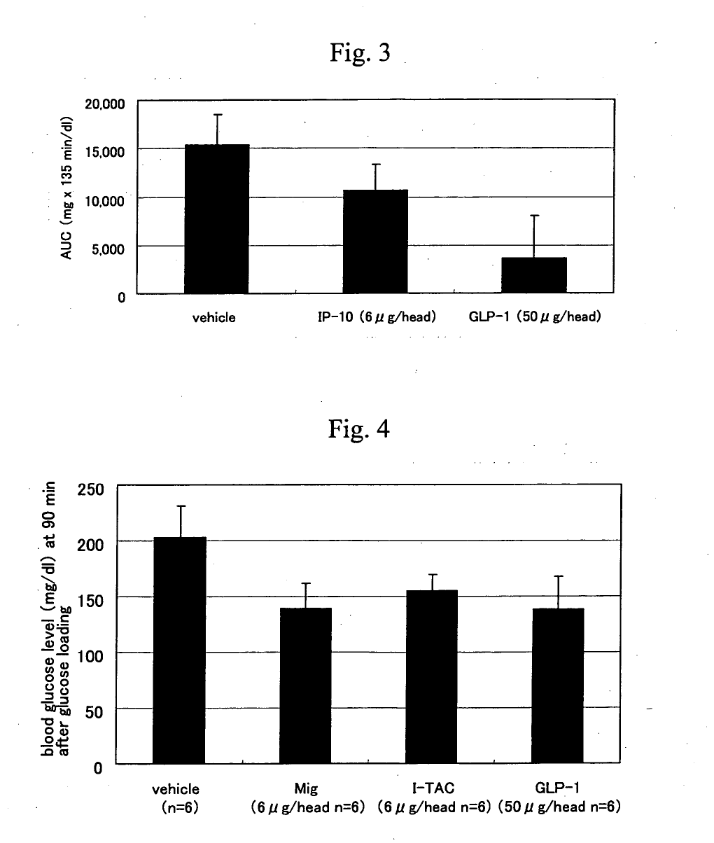

That is, 10 μl of blood was collected and mixed with 100 μL of 0.4 N perchloric acid, further 50 μL of 0.37 M potassium carbonate was added, and the supernatant after centrifugation was analyzed using the Glucose CII•Test Wako (glucose quantitation kit, mutarotase•GOD method, Wako Pure Chemicals) to determine the blood glucose level. As a result, IP-10 exhibited a glucose tolerance ameliorating effect nearly equivalent, on a dose basis, to that of GLP-1, which is a kind of incretin known to act on the pancreas to promote insulin secretion. Also, it was suggested from the changes in blood glucose level that insulin might be secreted in response to the transient elevation of blood glucose after glucose loading, and that insulin secreting action decreased as blood glucose decreased (FIGS. 2 and 3).

Also for Mig and I-TAC, which are CXCR3 ligands other than IP-10, mouse Mig or human I-TAC (both manufactured by BIOCARTA), previously dried, was dissolved in physiological saline (Otsuka Pharmaceutical), intravenous administration was conducted, and glucose tolerance ameliorating action was evaluated by the same technique. As a result, it was found that the blood glucose levels at 90 minutes after glucose loading in the Mig, I-TAC administration groups decreased compared to the solvent control group (FIG. 4). Hence, the CXCR3 ligands were shown to exhibit impaired glucose tolerance ameliorating action.

### EXAMPLE 2

### Establishment of CXCR3 Stably Expressing Line

\mathring{1} Construction of Gene-Transfection Vector

The coding region of CXCR3 is amplified by the PCR method using AmpliTaq (Perkin-Elmer). The amplified gene fragment is transfected downstream of a promoter that functions in animal cells (pcDAN4/HisMax (invitrogen)).

\mathring{2} Establishment of Cell Line

L1.2 cells are sown to a 10 cm² culture plate and cultured until a 60˜70% confluent state is reached. Subsequently, the medium is replaced with a serum-free medium and the CXCR3-transfected gene constructed in \mathring{1} above is allowed to form a complex with Lipofectamine-Plus (Gibco), after which the complex is added to the medium. After incubation for 5 hours, the medium is replaced with a medium containing 10% FBS, after which further cultivation is conducted for 8 hours. Subsequently, the cells are detached from the culture plate using trypsin EDTA, suspended in a medium containing G418 and 10% FBS, and sown to a 10 cm² culture plate. Several days later, the formed colony is isolated to yield a CXCR3 stably expressing line for CXCR3 ligand screening.

### EXAMPLE 3

### Screening of CXCR3 Ligands using FLIPR®

\mathring{1} Preparation of Cells

The CXCR3 stably expressing line for CXCR3 ligand screening is sown to a 96-well culture plate and cultured in a medium containing 10% FBS (Gibco) until a 60˜70% confluent state is reached.

\mathring{2} Quantitation of Intracellular Calcium by Fluorometry

The medium for the cells subjected to the above-described treatment is removed, a diluted compound to be evaluated (DMSO solution), 4 pM Fluo-3-AM (Teflab) and 2.5 mM probenecid are added, and cultivation is conducted at 37° C. for 60 minutes. The cells subjected to the above-described treatment are washed with ice-cooled PBS and suspended in Tyrode's medium (containing 2.5 mM probenecid and 1% gelatin). The absorptions in the culture plate at 488 nM and 540 nM are quantified using FLIPR® (Molecular Device).

### EXAMPLE 4

### Construction of Screening System for CXCR3 Ligand using FLIPR®

CHO/Gqi5 cells, which transiently or stably express CXCR3, were prepared and their responses to IP-10 and I-TAC, which are ligands, were confirmed.

(1) Materials

The cells used were CHO/Gqi5 cells (Molecular Devices), which were cultured with F-12 (Invirtogen), 10% FBS (ICN), 100 U/ml Penicillin, 100 μg/ml Streptomycin (Nacalai), and 250 μg/ml Hygromycin B (Invirtogen).

As the human CXCR3 expression plasmid, pcDNA3.1/Zeo-CXCR3 [prepared by amplifying the coding region of human CXCR3 by the PCR technique using AmpliTaq (Perkin-Elmer), and transfecting this to pcDNA3.1/Zeo (Invitrogen)] was used.

As the transfection reagent, LipofectAmine (Invirtogen) was used, and as the drug for selecting transformed cells, Zeocin (Invirtogen) was used.

As the reagents for FLIPR®, Fluo-3-AM (Molecular Probe), H/H/F/PB solution (Hanks' solution (Invirtogen), 20 mM HEPES, 0.1% FBS, 2.5 mM probenecid), and Pluronic acid (Molecular Probe) were used.

As the CXCR3 ligands, Human IP-10 (Peprotech) and human I-TAC (G/T) were used.

(2) Methods

1. Transient Gene Transfection to Cells

CHO/Gqi5 cells, at 2×10⁴ cells/well, were sown to a 96-well Black bottom clear plate (Corning), and cultured in the absence of antibiotics in a CO₂ incubator for 24 hours. 50 ng of pcDNA3.1/Zeo-CXCR3 or pcDNA3.1 was diluted in 5 μl of OPTI-MEM, and 0.3 μl of LipofectAmine was diluted in 5 μl of OPTI-MEM. Both were mixed and allowed to stand at room temperature for 30 minutes, after which 40 μl of a serum-free F12 medium was added, and the mixture was added to wells once washed with a serum-free F12 medium. After cultivation in a CO₂ incubator for 4 hours, the medium was replaced with a 10% FBS-supplemented F12 medium (once washed), and the cells were cultured in a CO₂ incubator for 24 hours and subjected to a measurement using FLIPR®.

2. Selection of Drug-Resistant Lines

1×10⁶ cells were sown to a T25 flask, and cultured in a CO₂ incubator for 24 hours. pcDNA3.1/Zeo-CXCR3 was cleaved with the restriction endonuclease SspI, and linearized at the vector portion. 2.5 μg of this plasmid DNA was diluted in 250 μl of OPTI-MEM, and 15 μl of LipofectAmine was diluted in 250 μl of OPTI-MEM. Both were mixed and allowed to stand at room temperature for 30 minutes, after which 2 ml of a serum-free F12 medium was added, and the mixture was added to a flask once washed with a serum-free F12 medium. After cultivation in a CO₂ incubator for 4 hours, the medium was replaced with a 10% FBS-supplemented F12 medium (once washed), and cultivation was conducted in a CO₂ incubator for 48 hours. The cells were recovered and sown to a 96-well plate at 1, 5 or 25 cells/well. At this time, Zeocin was added at 250 μg/ml. Three 96-well plates for each cell concentration, i.e., a total of nine plates, were cultured. About 20 days later, cells were recovered from the wells with evidence of cell proliferation, and sown to a 96-well Black bottom clear plate (Corning). Next day the plate was subjected to a measurement using FLIPR®, and selection of lines showing good ligand responsiveness was performed. For the lines showing good ligand responsiveness, recloning was performed. Each was selected at 0.1 cells/well on the 96-well plate, cells were recovered from the wells showing evidence of cell proliferation after about 20 days, and line selection was performed in the same manner as above.

3. Measurement of Calcium Signaling using FLIPR®

The cells recovered from each well were sown to a 96-well Black bottom clear plate (Corning). When cell counting was possible, the cells were sown at 2×10⁴ cells/well. Next day, the medium was removed, 50 μl of an H/H/F/PB solution containing 2 μM Fluo-3-AM and 0.02% Pluronic acid was added, and cultivation was conducted in a CO₂ incubator for 1 hour. The medium was replaced with 100 μl of H/H/F/PB solution and heating was conducted in a CO₂ incubator for 10 minutes, after which the plate was set to FLIPR® and further heated at 37° C. for 10 minutes, after which 25 μl of the ligand similarly heated at 37° C. was added using an injector, and a measurement of calcium signaling was conducted.

(3) Results

1. Confirmation of Ligand Responsiveness by Transient Transfection

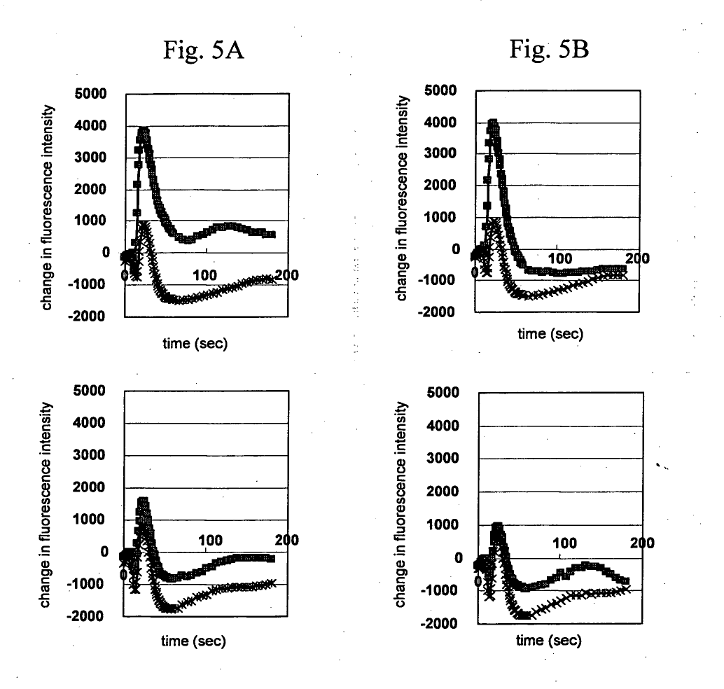

By the aforementioned method of (2)-1., pcDNA3.1/Zeo-CXCR3 or pcDNA3.1 was transiently transfected to CHO/Gqi5 cells, and responsiveness to IP-10 and I-TAC was confirmed using FLIPR® (FIG. 5). As a result, with either of the ligands IP-10 and I-TAC, a response was observed. On the other hand, when control pcDNA3.1 was transfected, no response was detected with either ligand.

2. Confirmation of Ligand Responsiveness in Stably Expressing Line

By the aforementioned method of (2)-2., pcDNA3.1/Zeo-CXCR3 was cleaved with the restriction endonuclease SspI and linearized at the vector portion. This was transfected to CHO/Gqi5 cells, and drug-resistant lines were selected using 250 μg/ml zeocin to obtain 156 drug-resistant lines. Next, for these resistant lines, responsiveness to IP-10 was confirmed using FLIPR®, and lines showing high ligand responsiveness (6 clones) were isolated. For these clones, recloning was performed, 16 clones for each of the obtained subclones were examined for ligand responsiveness, and finally #122-17 line was established as the CXCR3 expressing cell line.

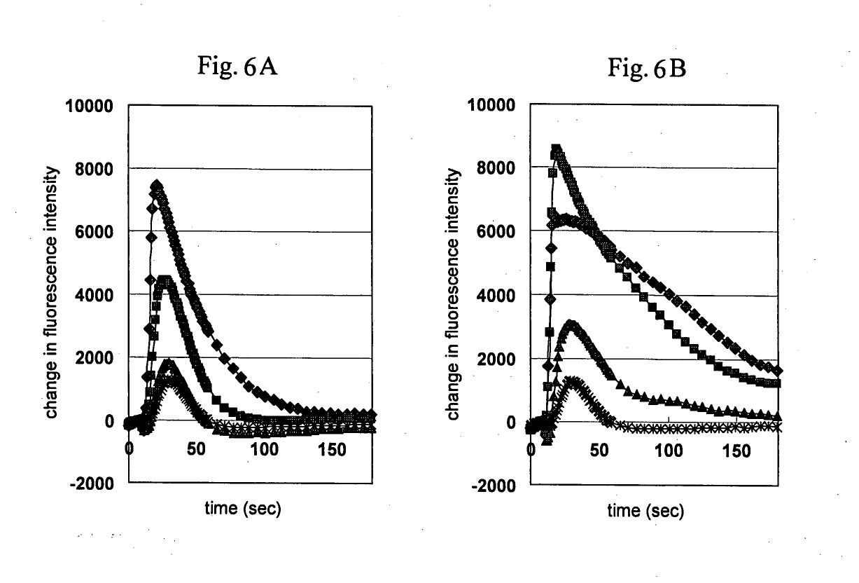

The concentration dependency of the responses of #122-17 line to IP-10 and I-TAC was examined (FIG. 6). As a result, for all ligands, concentration dependency could be confirmed; responsiveness could be confirmed from 300 ng/ml for IP-10, and from 100 ng/ml for I-TAC. By applying the test compound to the thus-prepared cell line, CXCR3 ligands can be screened.

### EXAMPLE 5

### Screening by Binding Assay

\mathring{1} Preparation of Cell Membrane Fraction

The CXCR3 stably expressing line for CXCR3 ligand screening or normal human monocytes or Th1 cells are sown to a flask and cultured in a medium containing 10% FBS (Gibco) until a 60˜70% confluent state is reached. The cells are recovered and suspended in buffer A (50 mM HEPES (pH 7.0), 10 mM 2-ME, 1 mM PMSF, 0.25 M sucrose). After homogenization using a Potter type homogenizer (400 rpm, 20 strokes), centrifugation is conducted at 100,000 g for 60 minutes, and the obtained precipitate is again suspended in buffer A. This suspension is overlain on 35% (mass/vol) sucrose in buffer A, and centrifugation is conducted at 45,000 g for 45 minutes. The interfacial fraction is recovered and suspended in buffer A, and centrifugation is conducted at 100,000 g for 60 minutes. The obtained precipitate is suspended in buffer A containing 20 μg/ml aprotinin and used in the assay below.

\mathring{2} Receptor Binding Assay

To MultiscreenGVPlate (Miripore), ¹²⁵I-labeled IP-10 (Amersham) suspended in a reaction buffer (50 mM Hepes pH 7.4, 12.5 mM magnesium acetate, 3.125 mM magnesium chloride, 0.125 mg/ml BSA) and a diluted compound to be evaluated (DMSO solution) are added. After incubation at 30° C., a membrane fraction prepared by the above-described procedures is added. After further incubation at 30° C., an equal amount of 20% TCA is added, the supernatant is removed by aspiration, and the protein is precipitated. After being washed with 10% TCA several times, the membrane is dried at 37° C. and punched out, and a measurement is taken using a γ counter.

### EXAMPLE 6

### Screening using BIACORE®

\mathring{1} Preparation of Cell Membrane Fraction

The CXCR3 stably expressing line for CXCR3 ligand screening or normal human monocytes or Th1 cells are sown to a flask and cultured in a medium containing 10% FBS (Gibco) until a 60˜70% confluent state is reached. The cells are recovered and suspended in buffer A (50 mM HEPES (pH 7.0), 10 mM 2-ME, 1 mM PMSF, 0.25 M sucrose). After homogenization using a Potter type homogenizer (400 rpm, 20 strokes), centrifugation is conducted at 100,000 g for 60 minutes, and the obtained precipitate is again suspended in buffer A. This suspension is overlain on 35% (mass/vol) sucrose in buffer A, and centrifugation is conducted at 45,000 g for 45 minutes. The interfacial fraction is recovered and suspended in buffer A, and centrifugation is conducted at 100,000 g for 60 minutes. The obtained precipitate is suspended in buffer A containing 20 μg/ml aprotinin and used in the assay below.

\mathring{2} Binding Measurement with BIACORE®

A common method described in the literature [Anal Biochem. 1998 Dec. 15; 265(2): 340-50. Markgren P O et al.] is used. CXCR3 (for example, 1-10 μg) prepared by the above-described procedures is dissolved in 10 mM acetate buffer (pH 4) and immobilized onto the matrix on the surface of the sensor chip CM5 of BIACORE® via a carboxyl group. The HBS buffer (manufactured by Amersham Pharmacia Biotech K.K.) is flown to the sensor chip at a flow rate of 20 μl/minute, and the background value is recorded. On the midway, the HBS buffer is replaced with the test compound dissolved in the HBS buffer at a concentration of 10 nM-10 M, the solution is flown for 1 minute, and the value change associated with the binding of the drug is recorded. Again, the solution is replaced with the drug-free HBS buffer, and the value change associated with the dissociation of the bound drug is recorded. The affinity between the test compound and CXCR3 is calculated from the binding and dissociation rates or the maximum amount bound.

### Industrial Applicability

Because the CXCR3 agonist of the present invention promotes insulin secretion in response to a transient elevation of blood glucose level and, after a reduction in blood glucose level, functions so that insulin secreting action decreases, it is potentially a safe impaired glucose tolerance ameliorating drug and therapeutic drug for diabetes without causing hypoglycemia. Also, because the CXCR3 antagonist of the present invention has an insulin secretion suppressing effect, it is useful as a therapeutic drug for hypoglycemia and other various diseases expected to be pathologically ameliorated by insulin secretion suppression, and as an anti-obesity drug.

Although the present invention has been described with emphasis on preferred embodiments, it is obvious to those skilled in the art that the preferred embodiments can be modified. The present invention intends that the present invention can be embodied by methods other than those described in detail in the present specification. Accordingly, the present invention encompasses all modifications encompassed in the gist and scope of the appended “CLAIMS.”

The present application is based on Japanese Patent Application No. 2002-101781 filed in Japan, the entire disclosure of which is included in the present specification. Also, the disclosures in all publications mentioned herein, including patents and patent application specifications, are incorporated by reference herein in the present invention to the extent that all of them have been given expressly.

1. A blood glucose and/or insulin secretion regulator comprising a CXCR3 ligand as an active ingredient.

2. An impaired glucose tolerance ameliorating drug comprising a CXCR3 agonist as an active ingredient.

3. The impaired glucose tolerance ameliorating drug of claim 2, wherein the CXCR3 agonist is at least one selected from the group consisting of IP-10, Mig, I-TAC, BCA-1, modifications thereof and prodrugs thereof.

4. The impaired glucose tolerance ameliorating drug of claim 2, wherein the CXCR3 agonist is at least one selected from the group consisting of IP-10, Mig, I-TAC, modifications thereof and prodrugs thereof.

5. The impaired glucose tolerance ameliorating drug of claim 2, which is a therapeutic drug for lifestyle-related diseases.

6. The impaired glucose tolerance ameliorating drug of claim 2, which is a therapeutic drug for diabetes.

7. A hypoglycemia ameliorating drug comprising a CXCR3 antagonist as an active ingredient.

8. A therapeutic drug for a disease that can be ameliorated by suppression of insulin secretion, which comprises a CXCR3 antagonist as an active ingredient.

9. An anti-obesity drug comprising a CXCR3 antagonist as an active ingredient.

10. A method of screening a CXCR3 ligand, which comprises bringing a test sample into contact with CXCR3 or a fragment thereof to which a ligand can bind, and selecting a compound that binds to said receptor or a fragment thereof.

11. A method of screening a CXCR3 ligand, which comprises bringing a known ligand into contact with CXCR3 or a fragment thereof to which a ligand can bind, in the presence and absence of a test substance, and comparing the binding activity of said receptor or fragment thereof and the known ligand under both conditions.

12. The screening method of claim 10, wherein CXCR3 or a fragment thereof to which a ligand can bind is provided in the form of a lipid bilayer embedding same.

13. A method of screening a CXCR3 ligand, which comprises comparing, in a reaction system comprising a CXCR3-containing lipid bilayer and an α-subunit of a G protein capable of being coupled with CXCR3, the GDP-GTP exchange reaction of said subunit or the cell stimulating activity of said G protein, in the presence and absence of a test substance.

14. The screening method of claim 13, wherein the reaction system is:
(i) a host eukaryotic cell transfected with an expression vector comprising a DNA that encodes CXCR3 and with an expression vector comprising a DNA that encodes the α subunit of a G protein capable of being coupled with CXCR3, (ii) a host eukaryotic cell transfected with an expression vector comprising a DNA that encodes a polypeptide consisting of CXCR3 and the α subunit of a G protein capable of being coupled with CXCR3 fused to the C-terminus side of CXCR3, (iii) a host animal cell that endogeneously expresses a G protein capable of being coupled with CXCR3, which is transfected with an expression vector comprising a DNA that encodes CXCR3, (iv) an animal cell that endogenously expresses CXCR3 and a G protein capable of being coupled with CXCR3, a homogenate of the cell, or a membrane fraction derived from the cell.

15. The screening method of claim 14, which comprises adding a GTP analogue to the reaction system in the presence and absence of a test substance, and comparing the binding of the α-subunit of the G protein capable of being coupled with CXCR3 and the GTP analogue under both conditions.

16. The screening method of claim 14, wherein the α-subunit of the G protein capable of being coupled with CXCR3 comprises a region that interacts with adenylate cyclase.

17. The method of claim 16, which comprises adding ATP to the reaction system in the presence and absence of a test substance, and comparing adenylate cyclase activity under both conditions.

18. The method of claim 16, which comprises comparing the cAMP amount in the cells of (i) to (iv) above, in the presence and absence of a test substance.

19. The screening method of claim 14, wherein the α-subunit of the G protein capable of being coupled with CXCR3 comprises a region that interacts with phospholipase Cβ.

20. The screening method of claim 19, which comprises adding phosphatidylinositol-4,5-diphosphate to the reaction system in the presence and absence of a test substance, and comparing phospholipase Cβ activity under both conditions.

21. The screening method of claim 19, which comprises comparing the amount of intracellular calcium ions in the cells of (i) to (iv) in the presence and absence of a test substance.

22. The screening method of claim 16, which comprises comparing the expression level of a reporter gene under the control of a promoter region comprising the cAMP-responsive element in the cells of (i) to (iv), which further comprise an expression vector containing said reporter gene, in the presence and absence of a test substance.

23. The screening method of claim 19, which comprises comparing the expression level of a reporter gene under the control of a promoter region comprising the TPA-responding element in the cells of (i) to (iv), which further comprise an expression vector containing said reporter gene, in the presence and absence of a test substance.

24. The screening method of claim 13, which is performed in the co-presence of a known ligand.

25. A blood glucose and/or insulin secretion regulator comprising a CXCR3 ligand selected by the method of claim 10 as an active ingredient.

26. An impaired glucose tolerance ameliorating drug comprising a CXCR3 agonist selected by the method of claim 10 as an active ingredient.

27. The impaired glucose tolerance ameliorating drug of claim 26, which is a therapeutic drug for lifestyle-related diseases.

28. The impaired glucose tolerance ameliorating drug of claim 26, which is a therapeutic drug for diabetes.

29. A hypoglycemia ameliorating drug comprising a CXCR3 antagonist selected by the method of claim 10 as an active ingredient.

30. A therapeutic drug for a disease that can be ameliorated by suppression of insulin secretion, which comprises a CXCR3 antagonist selected by the method of claim 10 as an active ingredient.

31. An anti-obesity drug comprising a CXCR3 antagonist selected by the method of claim 10 as an active ingredient.

32. A diagnostic method for type II diabetes, which comprises measuring a CXCR3 ligand or a transcript of a gene of said ligand in a biological sample using an antibody possessing specific affinity for the physiological ligand for CXCR3 or a nucleic acid that encodes said ligand or a nucleic acid hybridizable with said nucleic acid under stringent conditions.

33. A diagnostic reagent for type II diabetes, which comprises an antibody possessing specific affinity for a physiological ligand for CXCR3 or a nucleic acid that encodes said ligand or a nucleic acid hybridizable with said nucleic acid under stringent conditions.
